(19) United States
(12) Patent Application Publication
(10) Pub. No.: US 2026/0219272 A1
(43) Pub. Date: Jul. 30, 2026

(54) METHOD AND DEVICE FOR PREDICTING SIGNIFICANT GASTRIC ASPIRATION AND LUNG TRANSPLANT OUTCOME

(71) Applicant: UNIVERSITY HEALTH NETWORK, Toronto (CA)

(72) Inventors: Shafique KESHAVJEE, Toronto (CA); Marcelo CYPEL, Toronto (CA); Tereza MARTINU, Toronto (ON); Andrew SAGE, Toronto (CA); Rayoun RAMENDRA, Toronto (CA)

(21) Appl. No.: 19/182,347

(22) PCT Filed: Oct. 18, 2023
(86) PCT No.: PCT/CA2023/051383

§ 371 Date: Apr. 17, 2025

Related U.S. Application Data
(60) Provisional application No. 63/417,269, filed on Oct. 18, 2022.

(51) Int. Cl. G01N 33/573; G01N 33/543

(52) U.S. Cl. CPC G01N 33/573; G01N 33/54388; G01N 2333/96477; G01N 2800/245; G01N 2800/52

(57) ABSTRACT

Methods, kits and devices for detecting pepsinogen A4 (PGA4), and optionally bile acid (BA), in lungs such as transplant recipient lungs for assessing gastric aspiration and/or likelihood of developing an adverse outcome for the recipient are described. Methods involve obtaining from the lung a lung-derived fluid sample (LDFS); detecting in the sample the level, or presence or absence of PGA4, and optionally BA; and identifying the lung as i) likely to have gastric aspiration when PGA4 is present in LDFS, or the PGA4 level is greater than its cut-off in LDFS, and optionally when the level of the BA is greater than the BA cut-off level in LDFS, or ii) unlikely to have gastric aspiration when PGA4 is absent in LDFS, or the PGA4 level is equal or less than the PGA4 cut-off level in LDFS, and optionally when the BA level is equal or less than its cut-off in LDFS.

### RELATED APPLICATION

This application claims the benefit and priority of U.S. Provisional Patent Application 63/417,269 filed Oct. 18, 2022, herein incorporated by reference.

### FIELD

The disclosure pertains to methods, kits and devices for detecting pepsinogen A4 in donor lungs or transplanted recipient lungs for assessing the risk of adverse events.

### BACKGROUND

Despite the increasing number of lung transplants each year, approximately 75 to 80% of all donor lungs are declined for transplantation (1-3). The selection of the healthiest donor lungs is based on a number of clinical findings that include: donor type (brain vs cardiac death donors), age, smoking, ABGs, chest radiograph and bronchoscopic findings, and physical examination by the physician (1-3). One reason for declining donor lungs is the clinical suspicion of aspiration (4-5). Aspiration arises when gastric contents from the stomach that enter the lung (4-5). Aspiration can also lead to cell damage, death, and an advanced inflammatory response (4-5).

Aspiration of foregut contents into the tracheobronchial tree is a known risk factor for adverse outcomes in lung transplant (LTx) recipients. Bile acids detected in the airways can be used as a marker of aspiration, but they are secreted in the duodenum and likely reflect concurrent gastroparesis. There is currently no available marker specific to aspiration of solely contents from the stomach. Airway pepsin was studied as a marker of gastric aspiration, but it was not associated with reflux or adverse outcomes.

Following transplantation, gastroesophageal reflux disease (GERD) is a condition where patients experience chronic gastrointestinal content backwash to the esophagus, and lung transplantation could increase GERD in patients (12). GERD is known to cause microaspiration which exposes the lung to gastric contents and leads to chronic inflammation of lung tissues (13). Consequently, GERD has been considered as a risk factor for chronic lung allograft dysfunction (CLAD), hindering long-term survival in lung transplant patients (14). The current strategies for diagnosing GERD rely on the ambulatory pH monitoring in the distal esophagus during a 24-hr window (15). The limitations of pH monitoring include patient discomfort, limited time of observation and poor patient compliance.

The development of technologies such as rapid point-of-care technologies is desirable in order to enable for example a more accurate detection of aspiration and aid in the clinical decision-making processes for lung transplant patients.

### SUMMARY

The inventors herein disclose that the level or the presence of pepsinogen A4, and optionally bile acid, in a recipient as being associated with gastric aspiration in donor lungs and transplanted lungs. The inventors also disclose that the level or the presence of pepsinogen A4, and optionally bile acid as being associated with adverse outcomes in lung transplant recipients.

The disclosure provides in an aspect a method of assessing gastric aspiration in a lung, optionally a donor lung or a transplanted lung of a lung transplant recipient or from a lung of a subject with cystic fibrosis, emphysema, or pulmonary fibrosis or, or the likelihood of developing an adverse outcome in a lung transplant recipient, the method comprising:
a) obtaining a lung-derived fluid sample from the lung optionally donor lung or the transplanted lung;
b) detecting the level, or the presence or absence of pepsinogen A4 in the lung-derived fluid sample, and optionally detecting a level of a bile acid in the lung-derived fluid sample; and
c) identifying the lung as i) likely to have gastric aspiration when pepsinogen A4 is present in the lung-derived fluid sample, or the level of pepsinogen A4 is greater than the pepsinogen A4 cut-off level in the lung-derived fluid sample, and optionally when the level of the bile acid is greater than the bile acid cut-off level in the lung-derived fluid sample, or ii) unlikely to have gastric aspiration when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample.

In an embodiment, the bile acid cut-off level is determined by calculating in a population of lungs such as donor lungs an average bile acid cut-off level above which at least 90% of the donor lungs were identified as having gastric aspiration.

In an embodiment, when pepsinogen A4 is present in the lung-derived fluid sample from the lung, or the level of pepsinogen A4 is greater than the pepsinogen A4 cut-off level in the lung-derived fluid sample from the lung, the method described herein further comprises treating the subject for aspiration damage.

In an embodiment, when pepsinogen A4 is present in the lung-derived fluid sample from the transplanted lung, or the level of pepsinogen A4 is greater than the pepsinogen A4 cut-off level in the lung-derived fluid sample from the transplanted lung, the method described herein further comprises treating the transplanted lung for aspiration damage.

In an embodiment, when pepsinogen A4 is present in the lung-derived fluid sample from the transplanted lung, or the level of pepsinogen A4 is greater than the pepsinogen A4 cut-off level in the lung-derived fluid sample from the transplanted lung, and when the level of the bile acid in the lung-derived fluid sample from the transplanted lung is greater than the bile acid cut-off level, the method herein further comprises treating the transplanted lung for aspiration damage.

In an embodiment, when pepsinogen A4 is present in the lung-derived fluid sample from the donor lung, or the level of pepsinogen A4 is greater than the pepsinogen A4 cut-off level in the lung-derived fluid sample from the donor lung, the method described herein further comprises treating the transplanted lung for aspiration damage.

In an embodiment, when pepsinogen A4 is present in the lung-derived fluid sample from the donor lung, or the level of pepsinogen A4 is greater than the pepsinogen A4 cut-off level in the lung-derived fluid sample from the donor lung, and when the level of the bile acid in the lung-derived fluid sample from the donor lung is greater than the bile acid cut-off level, the method described herein further comprises treating the donor lung to reduce the likelihood of aspiration damage.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method further comprises performing EVLP in the donor lung to confirm lung function prior to transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises performing EVLP in the donor lung to confirm lung function prior to transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method described herein further comprises treating the donor lung with surfactant.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises treating the donor lung with surfactant.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method described herein further comprises declining the donor lung for transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises declining the donor lung for transplantation.

In an embodiment, the lung-derived fluid sample is a bronchoalveolar lavage (BAL) sample, a bronchial wash sample, an exhaled breath condensate sample, or an ex vivo lung perfusion (EVLP) perfusate sample.

In an embodiment, the bronchial wash sample is a large airway bronchial wash (LABW) sample.

In an embodiment, the bile acid is total bile acid (TBA) or a component thereof optionally selected from taurocholic acid (TCA), glycocholic acid (GCA) and cholic acid (CA).

In an embodiment, the bile acid is TBA.

In an embodiment, the dilution correction of the LABW sample is standardized by the amount of total protein, albumin, or urea.

In an embodiment, the pepsinogen A4 cut-off level is about 0.3 ng/uL. In an embodiment, the pepsinogen A4 cut-off level is about 0.312 ng/uL or about 0.313 ng/uL and a level of the pepsinogen A4 greater than the cut-off level is indicative the donor lung or transplanted lung is likely to have significant aspiration, and a level of pepsinogen A4 equal to or less than the cut-off level is indicative the donor lung or transplanted lung is unlikely to have 5 significant aspiration, and wherein the cut-off level is calculated by instilling and suctioning 20 mL of normal saline.

In an embodiment, the bile acid cut-off level is about 1245 nM and a level of the TBA greater than the cut-off level is indicative the donor lung as likely to have significant aspiration and a level of TBA equal to or less than the cut-off level is indicative the donor lung is unlikely to have had significant aspiration, and wherein the cut-off level is calculated by instilling and suctioning about 20 mL of normal saline.

In an embodiment, the method is carried out at point-of-care.

In an embodiment, the level, or the presence or absence of pepsinogen A4, and optionally the level of the bile acid, is detected by an immunoassay.

In an embodiment, the level, or the presence or absence of pepsinogen A4, and optionally the level of the bile acid, is detected by one or more of a Luminex® based assays, an enzyme-linked a immunosorbent assay (ELISA), immunofluorescence, radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, and any combination thereof.

In an embodiment, the adverse outcome is gastroesophageal reflux disease (GERD), chronic lung allograft dysfunction (CLAD), or death.

In an embodiment, the outcome is likely to develop aspiration pneumonia or other complication of gastric aspiration.

In an embodiment, the subject or recipient identified as likely to develop the adverse outcome of GERD is selected for anti-reflux surgery or medical reflux medications.

In an embodiment, the anti-reflux surgery is Nissen fundoplication or Toupet fundoplication.

In an embodiment, the recipient identified as likely to develop the adverse outcome of CLAD or death is selected for retransplant.

In an embodiment, the retransplanted recipient is assessed for the likelihood of developing an adverse outcome.

The disclosure also includes devices and kits for the detection of pepsinogen A4.

An aspect herein disclosed is a kit one or more detection agents specific for pepsinogen A4, and optionally one or more detection agents specific for the level of the bile acid, and instruction for use, the kit optionally further comprising one or more of a microtiter plate, an assay buffer, a wash buffer, a sample diluent, a standard diluent, a detection agent diluent, streptavidin-PE, a filter plate, a sealing tape, at least one standard, and any combination thereof, optionally for performing the methods herein disclosed.

Also provided is a method of assessing gastric aspiration of a subject, the method comprising:
a) obtaining a lung-derived fluid sample;
b) detecting a level, or the presence or absence, of pepsinogen A4 in the lung-derived fluid sample, and optionally detecting a level of a bile acid in the lung-derived fluid sample;
c) identifying a lung as i) likely to have gastric aspiration when pepsinogen A4 is present in the lung-derived fluid sample, or the level of pepsinogen A4 is greater than a pepsinogen A4 cut-off level in the lung-derived fluid sample, and optionally when the level of the bile acid is greater than a bile acid cut-off level in the lung-derived fluid sample, or ii) unlikely to have gastric aspiration when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample, or optionally iii) identifying the subject as having or likely to develop aspiration pneumonia, or having or likely to develop complications associated with gastric aspiration in a subject with cystic fibrosis, emphysema, or pulmonary fibrosis;
optionally wherein the bile acid cut-off level is determined by calculating in a population of healthy lungs an average bile acid cut-off level above which at least 90% of the lungs were identified as having gastric aspiration.

A point-of-care (POC) device for detecting the level, or presence or absence, of pepsinogen A4, and optionally for detecting the level of a bile acid, comprising:
a housing defining:
   a sample portion for receiving a fluid sample and a testing portion,
   a porous membrane connecting the sample portion to the testing portion, the porous membrane configured to draw the fluid sample from the sample portion to the testing portion; and
   one or more reagents or detection agents for pepsinogen A4, and optionally one or more reagents or detection agents for bile acid, the one or more reagents or detection agents being capable of reacting with the pepsinogen A4 to generate a detection signal, and optionally one or more reagents or detection agents for bile acid.

In an embodiment, the one or more reagents or detection agent are in fluid communication with the testing portion or immobilized on the testing portion. In an embodiment, the detection signal is a visible color change or a fluorescent signal.

In an embodiment, the device comprising a plurality of flow paths through which the sample passes and which distributes the sample to a plurality of testing zones within the testing portion.

In an embodiment, each of the testing zones comprises a different reagent or detection agent.

In an embodiment, the level, or the presence or absence, of pepsinogen A4, and optionally the level of the bile acid, is detected by an immunoassay.

In an embodiment, the level, or the presence or absence, of pepsinogen A4, and optionally the level of the bile acid, is detected by an immunoassay, comprising one or more of an enzyme-linked immunosorbent assay (ELISA), a multiplex assay, an electrochemical assay, an enzymatic assay, a colorimetric assay, and any combination thereof.

In an embodiment, the device is a lateral flow device.

In an embodiment, the fluorescent signal is detected by a reader.

In an embodiment, the device is for performing a method as described herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIG. 1 is a graph showing relative expression of PGC and PGA4 measured in large airway bronchial wash (LABW) of human lungs.

FIG. 2 is a graph showing LABW PGA4 levels pre- and post-anti-reflux surgery (n=61).

### DETAILED DESCRIPTION OF THE DISCLOSURE

### I. Definitions

The term “gastric aspiration disease or disorder” as used herein means a disease or disorder associated, related to, or is exacerbated by gastric aspiration, for example, but not limited to, aspiration pneumonia or other complications associated with gastric aspiration in a lung transplant recipient, or subject with cystic fibrosis, emphysema, or pulmonary fibrosis.

The term “lung-derived fluid” as used herein means a liquid obtained from lung airway(s). For example, the lung-derived fluid can be a bronchoalveolar lavage (BAL), a bronchial wash, an exhaled breath condensate, or an ex vivo lung perfusion (EVLP) perfusate. A lung-derived fluid sample can be obtained from a any subject or lung suspected of aspiration such as a donor lung or a transplanted lung, for example, obtaining the sample from a donor lung using ex vivo techniques, such as EVLP, or obtaining the sample using in vivo techniques from a lung transplant recipient after the recipient has received the transplanted lung.

The term “bronchial wash” as used herein means a liquid obtained by washing (or lavage) of lung airway(s). The washing comprises instilling and retrieving an amount of liquid (e.g. saline) through a bronchial airway for analysis. For example, the bronchial wash can be a large airway bronchial wash (LABW) or a bronchoalveolar lavage (BAL). Bronchial wash samples can be used to assess lung pathology and physiology. For example, as disclosed herein bronchial wash samples can be used to assess levels or the presence or absence of pepsinogen C, pepsinogen A4 and bile acid. A bronchial wash sample can be obtained from a lung ex vivo or in vivo e.g. a donor lung or transplanted lung or a lung in a subject with a lung condition or disorder where gastric aspiration can lead to complications.

The term “exhaled breath condensate” as used herein means a liquid from exhalation or a condensate of exhaled gas. The exhaled breath condensate comprises the collection of exhaled breath directed through, for example, a cooling device. As such, as disclosed herein exhaled breath condensate samples can be used to assess levels or the presence or absence of pepsinogen C, pepsinogen A4 and bile acid.

The term “EVLP” as used herein means ex vivo lung perfusion which is a technique to keep lung(s) alive outside of the body. This may allow a surgeon to evaluate the lungs for transplant suitability. EVLP mimics the environment of the lungs in the body. For example, the donor lungs are attached to a ventilator and filtration system, to deliver oxygen and nutrients, promote stability and remove bacteria, while being maintained at a steady temperature.

The term “aspiration” as used herein refers specifically to the condition known as gastric aspiration, where gastric content from the stomach enters the lung. Subjects at risk of gastric aspiration may include those who have undergone recent operations such as a lung transplant, those with impaired consciousness due to conditions like anesthesia or sedation, and those affected by neurological disorders such as Parkinson's disease, multiple sclerosis, or cerebral palsy. Additional risk factors affecting the severity of aspiration could include mechanical factors like intubation, advanced age, or physical disabilities affecting swallowing or coughing. Gastric aspiration can be assessed in donor lungs before harvest or after harvesting prior to transplant.

The term “significant aspiration” as used herein means a level of aspiration in a lung such as a donor lung or transplanted lung that increases the likelihood of developing an adverse outcome or for example renders a donor lung unsuitable for transplantation. Significant aspiration can be diagnosed clinically based on signs, symptoms, radiograph, or bronchoscopy, and it is associated with a significant increase in inflammatory biomarkers or significant reduction in lung function (>=10% drop in Forced expiratory volume (FEV1)). A donor lung may be unsuitable for transplant if for example PaO₂, levels are not suitable, if airway pressure is not suitable or if the lung the lung tissue is sufficiently damaged. PaO₂ is a metric in the donor lung for determining organ suitability for transplantation. Some other physiological features useful in determining suitability for transplantation include lung compliance and airway pressure(s). Significant aspiration can be assessed by measuring a level or the presence or absence of a bile acid and/or pepsinogen A4 in a lung-derived fluid sample. A cut-off level of pepsinogen A4 and/or bile acid can be selected according to for example a desired level of risk or particular patient characteristics and used to identify if a donor lung is likely to develop significant aspiration and therefore is unsuitable for transplantation, or a transplanted lung has significant aspiration and is likely to develop adverse outcomes.

The term “point-of-care” or “POC” as used herein means that testing using a method or device is performed or used near the potential lung donor or near the lung transplant recipient. For example, the testing does not have to be performed in a dedicated facility such as a pathology laboratory which may require trained technicians and professionals. “Point-of-care” testing can be accomplished through the use of transportable, portable and/or handheld devices and test kits (e.g. a point of care device). Advantages of point-of-care testing include the ability to obtain rapid analytical results, especially in emergency and/or time constrained situations. “Point-of-care devices” include, but are not limited to, lateral flow devices, lab-on-a child technologies, dipstick assay devices and colorimetric tests. The skilled person would understand that a point-of-care device can be developed based on the information provided in this disclosure, along with foundational knowledge in the field.

The term “PGA4” as used herein refers to pepsinogen A4 which is a precursor of the digestive enzyme pepsin, and includes all naturally occurring forms, for example from all species and particularly humans including for example human PGA4 which has amino acid sequence accession P0DJD7, herein incorporated by reference.

The term “biomarker” as used herein refers to pepsinogen A4 and optionally one or more bile acids. The term biomarker can also refer to a combination of bile acids, such as any combination of bile acids described herein or to TBA.

The term “bile acid” as used herein means total bile acid (TBA) or a component thereof for example selected from taurocholic acid (TCA), glycocholic acid (GCA) and cholic acid (CA). As it relates to TBA assay, TBA refers to the detection of common/conserved elements of any/all bile acids. A TBA assay can report levels of all bile acids, whether conjugated and unconjugated, as one value. The assay may also report levels individually. As used herein, “bile acid” includes conjugated and unconjugated bile acid. Conjugated bile acid is known in the art, for example TCA is a conjugated bile acid.

The term “polypeptide” as used herein refers to a polymer consisting of a number of amino acid residues bonded together in a chain. The polypeptide can form a part or the whole of a protein. The polypeptide can be arranged in a long, continuous and unbranched peptide chain. The polypeptide can also be arranged in a biologically functional way. The polypeptide can be folded into a specific three-dimensional structure that confers it a defined activity. The term “polypeptide” as used herein is used interchangeably with the term “protein”.

The term “cut-off level” as used herein refers a predetermined threshold value based on one or a plurality of known patient recipient outcomes, or with respect to biomarkers, levels above which are associated with increased level in poor outcomes or adverse events e.g. those identified as likely to have aspiration, adverse outcomes and/or not being suitable for transplant (e.g. rejected, further assessed with EVLP, declined following EVLP). For example, a level of a biomarker above the cut-off level can be associated with a donor lung or transplanted lung being identified as having an increased risk of developing aspiration or an increased risk of developing a negative outcome post-transplant and/or being declined after EVLP. Also, for example, a level of a biomarker below (and/or comparable to) the cut-off level can be associated with a candidate donor lung or transplanted lung being identified as having a decreased risk of developing aspiration and a decreased risk of developing a poor outcome post-transplant or being declined post EVLP. The threshold value can for example for each of the one or more biomarkers herein described, be determined from the levels or parameter values related thereto of the biomarkers in a plurality of known outcome lungs. For example, an optimal or an acceptable threshold can be selected based on the desired tolerable level of risk. The cut-off level can for example depend on the outcome being assessed. For example, for the patient outcome “ICU stay”, patients that have pre-transplant ICU stay are typically sicker and would also have longer post-transplant ICU stays. The cut-off level can be, for example, a level associated with duration of mechanical ventilation less than 72 h as a threshold in the ICU. The cut-off level can also for example be a value that is adjusted for or includes in some instances donor characteristics, for example gender, type (DBD or DCD), age, body mass index (BMI), and/or smoking history. Accordingly, the biomarker ‘cut-off’ can in some embodiments be adjusted for patients who have different duration of ICU stays, and donor characteristics. In an embodiment, the threshold is 72 h in the ICU. Multiple cut-off levels can also be employed. For example, there can be a first (low), second (medium) and third (high) cut off level. The “high” cut-off level, can for example be a predetermined level that is determined by calculating from a population that has clearly significant aspiration and identified for example as not suitable for transplantation and/or at risk of developing an adverse outcome. The medium cut-off level can for example be a predetermined level that is determined by calculating from a population that has average or an intermediate level of aspiration and for example potentially suitable for transplantation and/or at risk for developing an adverse outcome, for example in recipients that are less sick. The low cut-off level can be for example a predetermined level that is determined by calculating from a population that has slight or no aspiration and for example which lung would be suitable and/or there is low risk of developing an adverse outcome in most (e.g. greater than 90%) or all patients.

The term “suitable for transplant” as used herein means a lung that is predicted to be a good patient outcome lung graft, for example decreased risk of a prolonged ICU stay post-transplant, decreased time to extubation and/or decreased risk of developing CLAD, as compared to, for example, lungs with unsuitable pepsinogen A4, and optionally bile acid levels (e.g. predetermined by a cut-off level or as determined based on a population of lung transplant outcome and biomarker data). Levels of biomarker pepsinogen A4, and optionally bile acid can be used to assess whether the donor lung is suitable for transplant. For example, when a biomarker level from the donor lung (e.g. pepsinogen A4, and optionally bile acid such as TBA) is equal to or less than a pre-determined cut-off level, the donor lung is identified as suitable for transplant. “Suitable for transplant” means that the donor lung can be directly transplanted (e.g. without subjecting to EVLP) to a donor lung recipient. Alternatively, the donor lung can be further assessed by being subjected to EVLP and repeating assessment of the levels of pepsinogen A4, and optionally bile acid markers.

The term “unsuitable for transplant” as used herein means a lung that is predicted to be a negative or poor patient outcome lung graft, for example increased risk of prolonged ICU stay post-transplant, increased time to extubation and/or increased risk of developing CLAD as compared to, for example, lungs with low pepsinogen A4 and/or bile acid levels. Levels of biomarker pepsinogen A4, and optionally bile acid can be used to assess whether the donor lung is unsuitable for transplant. For example, when a biomarker level from the donor lung (e.g. pepsinogen A4, and optionally bile acid such as TBA) is greater than a pre-determined cut-off level, the donor lung is identified as unsuitable for transplant. Unsuitable lungs are either declined or can be further assessed by being subjected to EVLP.

The term “declined lungs” as used herein means lungs that after assessment and/or EVLP are declined for transplant. Such lungs can be discarded. Lungs are presently typically declined for example if gas exchange function is not acceptable, represented by a partial pressure of oxygen less than 350 mmHg with a fraction of inspired oxygen of 100%; or 15% worsening of lung compliance (represented by standard lung metric) compared to 1 h EVLP; or 15% worsening of pulmonary vascular resistance compared to 1 h EVLP; or development of significant edema; or worsening of ex vivo x-ray. In the present methods, lungs can be declined even if the gas exchange function is acceptable, there is no worsening of lung compliance or pulmonary vascular resistance, lack of edema or stable ex vivo x-ray if, for example, the pepsinogen A4 is present or its level is above an acceptable cut off level. Declining compliance indicates an injured lung; stable compliance is good and improves likelihood lung would be transplanted.

The term “clinical airway infection” as used herein refers to a pathological condition where there is an invasion and multiplication of harmful microorganisms, such as bacteria, viruses, or fungi, in the airways of an individual who has, for example, undergone a lung transplant. This condition is clinically important as it can lead to complications including bronchitis, pneumonia, and even acute or chronic rejection of the transplanted lung. The infection may manifest with symptoms such as fever, increased sputum production, changes in sputum color, shortness of breath, and lung function decline. Diagnosis can be made through clinical evaluation, imaging studies like chest X-rays or CT scans, and microbiological tests, such as sputum culture or bronchoalveolar lavage. Treatment usually involves the use of antibiotics, antivirals, or antifungals, depending on the causative agent, along with supportive care.

The term “antibody” as used herein is intended to include monoclonal antibodies including chimeric and humanized monoclonal antibodies, polyclonal antibodies, humanized antibodies, human antibodies, and chimeric antibodies. The antibody can be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques. The skilled person can readily recognize that a suitable antibody is any antibody useful for detecting pepsinogen A4 described herein in any detection method described herein. For example, useful antibodies include antibodies that specifically bind to pepsinogen A4.

The term “detection agent” refers to an agent, such as a detection antibody, that selectively binds and is capable of binding its cognate biomarker compared to another molecule and which can be used to detect a level and/or the presence of the biomarker. A biomarker specific detection agent can include probes and the like as well as binding polypeptides such as antibodies which can for example be used with Luminex® based assays, ELISA, immunofluorescence, radioimmunoassay, dot blotting, FACS, protein microarray, Western blots, immunoprecipitation followed by SDS-PAGE immunocytochemistry Simple Plex assay or Mass Spectrometry to detect the polypeptide level of a biomarker described herein. Similarly, “an antibody or fragment thereof” (e.g. binding fragment), that specifically binds a biomarker refers to an antibody or fragment that selectively binds its cognate biomarker compared to another molecule. “Selective” is used contextually, to characterize the binding properties of an antibody. An antibody that binds specifically or selectively to a given biomarker or epitope thereof can bind to that biomarker and/or epitope either with greater avidity or with more specificity, relative to other, different molecules. For example, the antibody can bind 3-5, 5-7, 7-10, 10-15, 5-15, or 5-30 fold more efficiently to its cognate biomarker compared to another molecule. The “detection agent” can for example be coupled to or labeled with a detectable marker. The label is preferably capable of producing, either directly or indirectly, a detectable signal. For example, the label can be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “level” as used herein refers to an amount (e.g. relative amount or concentration as well as parameter values calculable based thereon such as a rate or ratio) of biomarker (i.e. polypeptide related level) that is detectable, measurable or quantifiable in a test biological sample and/or a reference biological sample, for example, a BAL sample and/or a reference BAL sample. For example, the level can be a rate such as pg/mL/hour, a concentration such as μg/L, ng/ml or pg/mL, a relative amount or ratio such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, and/or 30 times more or less than a control biomarker or reference profile level. The control biomarker polypeptide level can, for example, be the average or median level in a plurality of known outcome lungs. Parameter values related to a level include concentration, rate of production and a ratio or fold increase (e.g. concentration at a later time point (such as 4 hours) divided by a concentration at an earlier time point for the same biomarker).

The term “subject” as used herein includes all members of the animal kingdom including mammals, and suitably refers to humans.

The term “limit of detection” (LOD) as used herein refers to a level above which a target (i.e. biomarker) can be confidently identified and/or quantified by a particular method of detection. For example, the LOD of an immunoassay such as ELISA can be 0.3 ng/uL, 0.312 ng/uL, or 0.313 ng/uL. The LOD can also be used as a cut-off level to determine the presence or absence of a biomarker, for example, pepsinogen A4.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods

Aspiration can have multiple physiological and pathological implications, leading to tissue damage and impaired function of the lungs. The presence of gastric material in the pulmonary system can trigger a cascade of cellular events, starting from cellular damage. If unaddressed, this can escalate to cell death and a heightened inflammatory response. Such cellular events compromise the lungs' ability to perform their primary function of gas exchange effectively, thereby negatively impacting overall lung health.

Disclosed herein is a method for detecting the level, or the presence or absence of biomarkers, for example pepsinogen A4 and optionally bile acid for assessing whether a lung such as a transplanted lung or donor lung has aspiration or likely to develop aspiration complications and/or whether a transplant lung recipient is at increased risk of GERD, CLAD or other adverse outcomes. More particularly, biomarkers (e.g. pepsinogen A4 and/or bile acid) are measured from lung-derived fluid samples such as bronchial wash samples, large airway bronchial wash sample, bronchoalveolar lavages samples, exhaled breath condensate samples or ex vivo lung perfusion perfusate samples. The biomarkers described herein, for example pepsinogen A4 and/or bile acid can be used as a point-of-care (POC) screening tool, facilitating early assessment and/or triage of transplant recipient lungs and donor lungs and allowing physicians to better prevent adverse outcomes. The methods can also be used in subjects that have a lung disease or disorder such as cystic fibrosis, emphysema or pulmonary fibrosis, where aspiration can be of greater risk, and cause complications such as aspiration pneumonia. POC devices herein disclosed can also help in determining the occurrence of pulmonary aspiration, which can enable early diagnostic and management of for example conditions associated with gastric aspiration, CLAD and/or GERD post-transplant to minimize allograft injury and chronic lung inflammation. The methods disclosed herein can be qualitative, semi-quantitative, or quantitative. For example, qualitative evaluation can be a colorimetric assay where the result can be assessed visually, as in the detection of the presence or absence of the line in a pregnancy test. Semi-quantitative evaluation can, for example, based on the number and the intensity of multiple test lines (see e.g., Parolo C et al., Tutorial: design and fabrication of nanoparticle-based lateral-flow immunoassays. Nat Protoc 15, 3788-3816 (2020), the content of which is incorporated by reference herein in its entirety).

Accordingly, one aspect of the present disclosure is a method of assessing gastric aspiration in a lung optionally a lung from a subject with cystic fibrosis, emphysema, or pulmonary fibrosis, a donor lung or a transplanted lung of a lung transplant recipient, or the likelihood of developing an adverse outcome in a lung transplant recipient, the method comprising:
a) obtaining a lung-derived fluid sample from the lung, optionally a lung from a subject with cystic fibrosis, emphysema, or pulmonary fibrosis, donor lung or transplanted lung;
b) detecting the level, or the presence or absence, of pepsinogen A4 in the lung-derived fluid sample, and optionally detecting a level of a bile acid in the lung-derived fluid sample; and
c) identifying the lung as i) likely to have gastric aspiration when pepsinogen A4 is present in the lung-derived fluid sample, or the level of pepsinogen A4 is greater than the pepsinogen A4 cut-off level in the lung-derived fluid sample, and optionally when the level of the bile acid is greater than the bile acid cut-off level in the lung-derived fluid sample, or ii) unlikely to have gastric aspiration when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample, optionally wherein the bile acid cut-off level is determined by calculating in a population of lungs, optionally donor lungs an average bile acid cut-off level above which at least 90% of the donor lungs were identified as having gastric aspiration.

In an embodiment, when pepsinogen A4 is present in the lung-derived fluid sample from the lung, or the level of pepsinogen A4 is greater than the pepsinogen A4 cut-off level in the lung-derived fluid sample from the lung, the method described herein further comprises treating the lung for aspiration damage.

In an embodiment, when pepsinogen A4 is present in the lung-derived fluid sample from the transplanted lung, or the level of pepsinogen A4 is greater than the pepsinogen A4 cut-off level in the lung-derived fluid sample from the transplanted lung, the method described herein further comprises treating the transplanted lung for aspiration damage.

In an embodiment, when pepsinogen A4 is present in the lung-derived fluid sample from the lung, or the level of pepsinogen A4 is greater than the pepsinogen A4 cut-off level in the lung-derived fluid sample from the lung, and when the level of the bile acid in the lung-derived fluid sample from the lung is greater than the bile acid cut-off level, the method herein further comprises treating the lung for aspiration damage.

In an embodiment, when pepsinogen A4 is present in the lung-derived fluid sample from the transplanted lung, or the level of pepsinogen A4 is greater than the pepsinogen A4 cut-off level in the lung-derived fluid sample from the transplanted lung, and when the level of the bile acid in the lung-derived fluid sample from the transplanted lung is greater than the bile acid cut-off level, the method herein further comprises treating the transplanted lung for aspiration damage.

In an embodiment, when pepsinogen A4 is present in the lung-derived fluid sample from the donor lung, or the level of pepsinogen A4 is greater than the pepsinogen A4 cut-off level in the lung-derived fluid sample from the donor lung, the method described herein further comprises treating the donor lung to reduce the likelihood of aspiration damage.

In an embodiment, when pepsinogen A4 is present in the lung-derived fluid sample from the donor lung, or the level of pepsinogen A4 is greater than the pepsinogen A4 cut-off level in the lung-derived fluid sample from the donor lung, and when the level of the bile acid in the lung-derived fluid sample from the donor lung is greater than the bile acid cut-off level, the method described herein further comprises treating the donor lung to reduce the likelihood of aspiration damage.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method further comprises performing EVLP in the donor lung to confirm lung function prior to transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises performing EVLP in the donor lung to confirm lung function prior to transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method described herein further comprises treating the donor lung with surfactant.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises treating the donor lung with surfactant.

In an embodiment, the surfactant comprises beractant, calfactant, poractant alfa, lucinactant, and/or a synthetic surfactant. In an embodiment, the synthetic surfactant is ExoSurf™. In an embodiment, the surfactant comprises bovine lung extract surfactant (BLES). In an embodiment, surfactant treatment comprises a composition comprising phospholipid and BLES. In an embodiment, the composition comprises about 54 mg phospholipid per about 2 mL of surfactant. In an embodiment, the composition comprises about 54 mg phospholipid per about 2 mL BLES. In an embodiment, the surfactant treatment comprises administering the composition comprising about 18 mg to about 54 mg phospholipid per kg body weight. In embodiment, the surfactant treatment comprises administering the composition comprising about 18 mg phospholipid and about 0.67 mL BLES, to about 54 mg phospholipid and about 2 mL BLES, per kg body weight. In embodiment, the surfactant treatment comprises administering the composition comprising about 27 mg phospholipid and about 1 mL BLES, to about 54 mg phospholipid and about 1.67 mL BLES, per kg body weight. In an embodiment, the surfactant treatment comprises administering composition comprising about 36 mg phospholipid per kg body weight. In an embodiment, the surfactant treatment comprises administering composition comprising about 36 mg phospholipid and about 1.33 mL BLES per kg body weight. In an embodiment, the surfactant or a composition comprising the surfactant is administered into each segment via bronchoscopy.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, the method described herein further comprises declining the donor lung for transplantation.

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and when the level of the bile acid in the lung-derived fluid sample from the donor lung is greater than the bile acid cut-off level, the method described herein further comprises declining the donor lung for transplantation.

The lung-derived fluid sample can be sourced from a number of different types of samples, for example, it can be a bronchoalveolar lavage (BAL) sample, a bronchial wash sample, an exhaled breath condensate sample, or an ex vivo lung perfusion (EVLP) perfusate sample. In an embodiment, the lung-derived fluid sample is a BAL sample, a bronchial wash sample, an exhaled breath condensate sample, or an ex vivo lung perfusion (EVLP) perfusate sample. In an embodiment, the lung-derived fluid sample is a bronchoalveolar lavage (BAL) sample. In an embodiment, the lung-derived fluid sample is a bronchial wash sample. In an embodiment, the lung-derived fluid sample is an exhaled breath condensate sample. In an embodiment, the lung-derived fluid sample is an ex vivo lung perfusion (EVLP) perfusate sample. In an embodiment, the bronchial wash sample is a large airway bronchial wash (LABW) sample.

The bile acid described herein can comprise total bile acid (TBA), or a component thereof selected from taurocholic acid (TCA), glycocholic acid (GCA), cholic acid (CA), and combinations thereof. In an embodiment, the bile acid is total bile acid (TBA). In an embodiment, the bile acid is selected from one or more of taurocholic acid (TCA), glycocholic acid (GCA), cholic acid (CA), and combinations thereof. In an embodiment, the lung derived fluid sample is a bronchoalveolar lavage sample obtained from a transplanted lung or a donor lung. In an embodiment, the lung derived fluid sample is a bronchial wash sample obtained from a transplanted lung or a donor lung. In an embodiment, the lung derived fluid sample is an exhaled breath condensate sample obtained from a transplanted lung or a donor lung. In an embodiment, the EVLP perfusate sample is obtained from a donor lung.

In an embodiment, to facilitate comparison between samples dilution correction of the LABW sample is standardized by the amount of total protein, albumin, or urea. The dilution correction normalizes the detected biomarker, for example pepsinogen A4, relative to the concentration of total protein, albumin or urea of the LABW sample. In an embodiment, the total level of pepsinogen A4 is normalized to the total protein concentration of the LABW sample to facilitate comparison between samples. In an embodiment, the level of pepsinogen A4 is normalized to the albumin concentration of the LABW sample to facilitate comparison between samples. In an embodiment, the level of pepsinogen A4 is normalized to the urea concentration of the LABW sample to facilitate comparison between samples. In an embodiment, the total level of pepsinogen A4 and bile acid is normalized to the total protein concentration of the LABW sample to facilitate comparison between samples. In an embodiment, the level of pepsinogen A4 and bile acid is normalized to the albumin concentration of the LABW sample to facilitate comparison between samples. In an embodiment, the level of pepsinogen A4 and bile acid is normalized to the urea concentration of the LABW sample to facilitate comparison between samples.

The sample may be centrifuged and the supernatant assessed, optionally directly (e.g. without concentration). The supernatant or sample may be frozen. The supernatant or sample may be concentrated.

In an embodiment, the lung-derived fluid sample is a bronchial wash sample obtained in accordance with the International Society for Heart and Lung Transplantation Consensus Statement for the Standardization of Bronchoalveolar Lavage in Lung Transplantation (Martinu et al. J Heart Lung Transplant, “International Society for Heart and Lung Transplantation consensus statement for the standardization of bronchoalveolar lavage in lung transplantation”, 2020 November; 39(11):1171-1190; PMID: 32773322 the content of which is incorporated by reference herein in its entirety).

In an embodiment, the lung-derived fluid sample is obtained before retrieval (i.e. while the lung is still in the body of the donor). In an embodiment, the lung-derived fluid sample is obtained shortly after retrieval from donor, for example 0.5, 1, 2, 4, 6, 8, 10 or 12 hours following retrieval. In some embodiments, the lung-derived fluid sample is obtained before EVLP or during EVLP. In some embodiments, the lung-derived fluid sample is obtained after 0.5, 1, 2, 3, 4, 5 or 6 hours of EVLP.

In an embodiment the adverse outcome is gastroesophageal reflux disease (GERD).

In an embodiment the adverse outcome is chronic lung allograft dysfunction (CLAD).

In an embodiment the adverse outcome is death.

In an embodiment, the adverse outcome is aspiration pneumonia.

In an embodiment, the recipient identified as likely to develop the adverse outcome of GERD is selected for anti-reflux surgery or medical reflux medications.

In an embodiment, the anti-reflux surgery is Nissen fundoplication or Toupet fundoplication.

In an embodiment, the recipient identified as likely to develop the adverse outcome of CLAD or death is selected for re-transplant.

In an embodiment, the re-transplanted recipient is assessed for the likelihood of developing an adverse outcome.

In an embodiment, the method is carried out at point-of-care.

The levels of pepsinogen A4 and optionally bile acid can be detected using a number of methods. In an embodiment, the level or the presence or absence, of pepsinogen A4, is detected by immunoassay. In an embodiment, the level of pepsinogen A4 is detected by immunoassay. In an embodiment, the presence or absence of pepsinogen A4 is detected by immunoassay. In an embodiment, the level or the presence or absence of pepsinogen A4, and the level of bile acid, are detected by immunoassay. In an embodiment, the level of pepsinogen A4 and the level of bile acid are detected by immunoassay. In an embodiment, the presence or absence of pepsinogen A4, and the level of bile acid, are detected by immunoassay. In an embodiment, pepsinogen A4 is detected or determined by one or more of Luminex® based assays, an enzyme-linked immunosorbent assay (ELISA), immunofluorescence, a radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, or any combination thereof. In an embodiment, pepsinogen A4 and bile acid are detected or determined by one or more of Luminex® based assays, an enzyme-linked immunosorbent assay (ELISA), immunofluorescence, a radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, or any combination thereof. In an embodiment, the level or presence of pepsinogen A4 is detected by an ELISA. In an embodiment, the level or presence of pepsinogen A4, and the level of bile acid, are detected by an ELISA. In an embodiment, the level or the presence or absence of pepsinogen A4 is detected by an ELISA as described in Example 1. In an embodiment, the level of pepsinogen A4 is detected by an ELISA, as described in Example 1. In an embodiment, the presence or absence of pepsinogen A4 is detected by an ELISA, as described in Example 1. In an embodiment, the level or the presence or absence of pepsinogen A4 is detected by an ELISA as described in Example 2B. In an embodiment, the level of pepsinogen A4 is detected by an ELISA, as described in Example 2B. In an embodiment, the presence or absence of pepsinogen A4 is detected by an ELISA as described in Example 2B.

In an embodiment, the level of bile acid is detected using an immunoassay. In an embodiment, the level of bile acid is detected by an ELISA. In an embodiment, the level or presence of pepsinogen A4, and the level of bile acid, are detected using an immunoassay. In an embodiment, the level or presence of pepsinogen A4, and the level of bile acid, are detected by at least one ELISA.

In an embodiment, the level or the presence or absence of pepsinogen A4 is detected using mass spectrometry. In an embodiment, the level of pepsinogen A4 is detected using mass spectrometry. In an embodiment, the presence or absence of pepsinogen A4 is detected using mass spectrometry. In an embodiment, the level of bile acid is detected by mass spectrometry. In an embodiment, the level or the presence or absence of pepsinogen A4, and the level of bile acid, are detected using mass spectrometry. In an embodiment, the level or presence of pepsinogen A4 is detected using immunofluorescence. In an embodiment, the level of bile acid is detected by immunofluorescence. In an embodiment, the level or presence of pepsinogen A4, and the level of bile acid, are detected using immunofluorescence.

In an embodiment, the level or presence of pepsinogen A4 is detected using a radioimmunoassay. In an embodiment, the level of bile acid is detected by a radioimmunoassay. In an embodiment, the level or presence of pepsinogen A4, and the level of bile acid, are detected using a radioimmunoassay.

In an embodiment, the level or presence of pepsinogen A4 is detected using dot blotting. In an embodiment, the level of bile acid is detected by dot blotting. In an embodiment, the level or presence of pepsinogen A4, and the level of bile acid, are detected using dot blotting.

In an embodiment, the level or presence of pepsinogen A4 is detected using FACS. In an embodiment, the level of bile acid is detected by FACS. In an embodiment, the level or presence of pepsinogen A4, and the level of bile acid, are detected using FACS.

In an embodiment, the level or presence of pepsinogen A4 is detected using a protein microarray. In an embodiment, the level of bile acid is detected by a protein microarray. In an embodiment, the level or presence of pepsinogen A4, and the level of bile acid, are detected using a protein microarray.

In an embodiment, the level or presence of pepsinogen A4 is detected using immunoprecipitation followed by SDS-PAGE. In an embodiment, the level of bile acid is detected by immunoprecipitation followed by SDS-PAGE. In an embodiment, the level or presence of pepsinogen A4, and the level of bile acid, are detected using immunoprecipitation followed by SDS-PAGE.

In an embodiment, the level or presence of pepsinogen A4 is detected using immunocytochemistry. In an embodiment, the level of bile acid is detected by immunocytochemistry. In an embodiment, the level or presence of pepsinogen A4, and the level of bile acid, are detected using immunocytochemistry.

In an embodiment, the level or presence of pepsinogen A4 is detected using a simple Plex assay. In an embodiment, the level of bile acid is detected by a simple Plex assay. In an embodiment, the level or presence of pepsinogen A4, and the level of bile acid, are detected using a simple Plex assay.

In an embodiment, the level of or presence pepsinogen A4 is detected using a multiplex assay. In an embodiment, the level of bile acid is detected by a multiplex assay. In an embodiment, the level or presence of pepsinogen A4, and the level of bile acid, are detected using a multiplex assay.

In an embodiment, the level or presence of pepsinogen A4 is detected using an electrochemical assay. In an embodiment, the level of bile acid is detected by an electrochemical assay. In an embodiment, the level or presence of pepsinogen A4, and the level of bile acid, are detected using an electrochemical assay.

In an embodiment, the level or presence of pepsinogen A4 is detected using an enzymatic assay. In an embodiment, the level of bile acid is detected by an enzymatic assay. In an embodiment, the level or presence of pepsinogen A4, and the level of bile acid, are detected using an enzymatic assay.

In an embodiment, the level or presence of pepsinogen A4 is detected using a colorimetric assay. In an embodiment, the level of bile acid is detected by a colorimetric assay. In an embodiment, the level or presence of pepsinogen A4, and the level of bile acid, are detected using a colorimetric assay.

In an embodiment, the level or presence of pepsinogen A4 is detected using Luminex® based assays. In an embodiment, the level of bile acid is detected by Luminex® based assays. In an embodiment, the level or presence of pepsinogen A4, and the level of bile acid, are detected using Luminex® based assays.

In an embodiment, the risk of developing an adverse outcome is assessed relative to a control.

In an embodiment, the control is a healthy lung. In an embodiment, the control is a transplanted lung or donor lung that has not experienced aspiration or an adverse outcome. In an embodiment, the control is a population of healthy lungs. In an embodiment, the control is a population of transplanted lungs and/or donor lungs that have not experienced aspiration or an adverse outcome.

In an embodiment, pepsinogen A4 is not detected in the control healthy lung.

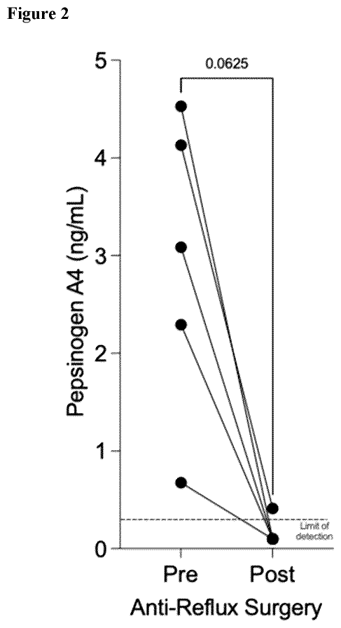

In an embodiment, the limit of detection (LOD) of pepsinogen A4 is below the level of detection in an ELISA, as shown in FIG. 2.

The LOD described herein can be used as cut-off level for assessing aspiration such as gastric aspiration, and the likelihood of a transplanted lung recipient in developing an adverse outcome. This LOD can be applied in, for example, LABW samples. The LOD is in an ELISA is the lowest concentration of the analyte, for example pepsinogen A4, that can be reliably distinguished from the background signal or noise. Deriving the LOD can involve steps that include i) preparing a series of dilutions of the known standard analyte where the dilutions serve as the standard curve; ii) running the known standards and blanks, where the blanks contain all the components except for the analyte; iii) generating a standard curve by plotting the absorbance or fluorescence values of the known standards against their concentrations to generate the standard curve, usually by fitting the data to a linear or logistic regression model; iv) measuring background signal by which the absorbance or fluorescence of the blanks is measured to determine the background signal; v) calculating mean and standard deviation of the background signal; vi) determining the LOD which is typically the mean background signal plus two or three standard deviations, depending on the desired level of confidence; vii) verifying LOD, for example, by running samples with known concentrations around the calculated LOD to verify its accuracy, and to ensure that the signal at the LOD is statistically different from the background signal; and viii) analyzing data to confirm that the calculated LOD allows for reliable detection of the analyte, and statistical methods such as regression analysis can be applied to validate the LOD. The skilled person would recognize these steps could be modified or omitted, or additional steps can be added, so long that the LOD can be used to reliably distinguish a signal from noise.

In an embodiment, the pepsinogen A4 cut-off level is about 0.3 ng/uL. In an embodiment, the limit of detection (LOD) of pepsinogen A4 by ELISA is about 0.312 ng/uL or about 0.313 ng/ml. In an embodiment, the ELISA is Human Pepsinogen A4 DuoSet ELISA from R&D Systems. In an embodiment, the pepsinogen A4 cut-off level is about 0.3 ng/uL. In an embodiment, the pepsinogen A4 cut-off level by ELISA is about 0.312 ng/uL or about 0.313 ng/uL. In an embodiment, the pepsinogen A4 cut-off level by ELISA is about 0.312 ng/uL or about 0.313 ng/uL and a level of the pepsinogen A4 greater than the cut-off level is indicative the donor lung or transplanted lung is likely to have significant aspiration, and a level of pepsinogen A4 equal to or less than the cut-off level is indicative the donor lung or transplanted lung is unlikely to have significant aspiration, and wherein the cut-off level is calculated by instilling and suctioning 20 mL of normal saline. The skilled person recognizes that the volume suctioned is less than the volume instilled, because some of the fluid (e.g. 10%, 15% or 20%) gets lost in the airways. Different cut off levels can be calculated for different volumes. Different cut off levels can be calculated depending on how the sample or supernatant is processed.

In an embodiment, the bile acid cut-off level is about 1245 nM and a level of the TBA greater than the cut-off level is indicative the donor lung as likely to have significant aspiration and a level of TBA equal to or less than the cut-off level is indicative the donor lung is unlikely to have had significant aspiration, and wherein the cut-off level is calculated by instilling and suctioning 20 mL of normal saline. The skilled person recognizes that the volume suctioned is less than the volume instilled, because some of the fluid (e.g. 10%, 15% or 20%) gets lost in the airways. Different cut off levels can be calculated for different volumes. Different cut off levels can be calculated depending on how the sample or supernatant is processed.

In another aspect, also provided is a method of assessing gastric aspiration of a subject, the method comprising:
a) obtaining a lung-derived fluid sample;
b) detecting a level, or the presence or absence, of pepsinogen A4 in the lung-derived fluid sample, and optionally detecting a level of a bile acid in the lung-derived fluid sample;
c) identifying a lung as i) likely to have gastric aspiration when pepsinogen A4 is present in the lung-derived fluid sample, or the level of pepsinogen A4 is greater than a pepsinogen A4 cut-off level in the lung-derived fluid sample, and optionally when the level of the bile acid is greater than a bile acid cut-off level in the lung-derived fluid sample, or ii) unlikely to have gastric aspiration when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample, or optionally iii) identifying the subject as having or likely to develop aspiration pneumonia, or having or likely to develop complications associated with gastric aspiration in a subject with cystic fibrosis, emphysema, or pulmonary fibrosis;
optionally wherein the bile acid cut-off level is determined by calculating in a population of healthy lungs an average bile acid cut-off level above which at least 90% of the lungs were identified as having gastric aspiration.

In an embodiment, the subject is at risk of aspiration due to recent operation, impaired consciousness, for example due to anesthesia, sedation, stroke, neurological disorders such as Parkinson's disease, multiple sclerosis, or cerebral palsy. In an embodiment, the subject is at risk of aspiration due to recent operation. In an embodiment, the subject is at risk of aspiration due to impaired consciousness. In an embodiment, the impaired consciousness is due to anesthesia. In an embodiment, the subject is at risk of aspiration due to sedation. In an embodiment, the subject is at risk of aspiration due to stroke.

In an embodiment, the subject is at risk of aspiration due to neurological disorders. In an embodiment, the neurological disorder is Parkinson's disease. In an embodiment, the neurological disorder is multiple sclerosis. In an embodiment, the neurological disorder is cerebral palsy.

Additional risk factors affecting the severity of aspiration could include mechanical factors like intubation, advanced age, or physical disabilities affecting swallowing or coughing. In an embodiment, the subject is at risk of aspiration due to a mechanical factor. In an embodiment, the subject is undergoing intubation. In an embodiment, the subject is of advanced age. In an embodiment, the subject has a physical disability affecting swallowing or coughing.

In an embodiment, the lung is from a subject with cystic fibrosis, emphysema, pulmonary fibrosis, a donor lung prior to or after lung transplantation, or a lung of a patient who has undergone a surgery or intervention, optionally a lung surgery. In an embodiment, the lung surgery is lobectomy, segmentectomy, wedge resection, thoracotomy, thoracoscopic surgery, bullectomy, pleurectomy, decortication, tracheostomy, bung volume reduction surgery, sleeve resection, pulmonary metastasectomy, or lung transplant. In an embodiment, the lung surgery is lung transplant. In an embodiment, the intervention is an intervention associated with bronchoscopy.

In an embodiment, when pepsinogen A4 is present in the lung-derived fluid sample from a transplanted lung, or the level of pepsinogen A4 is greater than the pepsinogen A4 cut-off level in the lung-derived fluid sample from the transplanted lung, and optionally when the level of the bile acid in the lung-derived fluid sample from the transplanted lung is greater than the bile acid cut-off level, the method described herein further comprises treating the lung to reduce the likelihood of aspiration damage.

In an embodiment, when pepsinogen A4 is present in the lung-derived fluid sample from a donor lung, or the level of pepsinogen A4 is greater than the pepsinogen A4 cut-off level in the lung-derived fluid sample from the donor lung, and optionally when the level of the bile acid in the lung-derived fluid sample from the donor lung is greater than the bile acid cut-off level, the method described herein further comprises treating the donor lung to reduce the likelihood of aspiration damage;

In an embodiment, when pepsinogen A4 is present in the lung-derived fluid sample from a donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises performing EVLP on the donor lung to confirm lung function prior to transplantation;

In an embodiment, when pepsinogen A4 is present in the lung-derived fluid sample from the donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises treating the donor lung with surfactant. In an embodiment, the surfactant comprises beractant, calfactant, poractant alfa, lucinactant, and/or a synthetic surfactant. In an embodiment, the synthetic surfactant is ExoSurf™.

In an embodiment, when pepsinogen A4 is present in the lung-derived fluid sample from a donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises declining the donor lung for transplantation.

In an embodiment, when pepsinogen A4 is present in the lung-derived fluid sample from a lung, or the level of pepsinogen A4 from the lung-derived fluid sample from the subject lung is greater than the pepsinogen A4 cut-off level, and optionally when the level of the bile acid in the lung-derived fluid sample from the subject lung is greater than the bile acid cut-off level, the method described further comprises treating the subject with complications associated with gastric aspiration.

In an embodiment, the lung-derived fluid sample is a bronchoalveolar lavage (BAL) sample, a bronchial wash sample, an exhaled breath condensate sample, or an ex vivo lung perfusion (EVLP) perfusate sample.

In an embodiment, the bronchial wash sample is large airway bronchial wash (LABW) sample.

In an embodiment, the method comprises detecting the level of a bile acid in the lung-derived fluid sample, wherein the bile acid is total bile acid (TBA) or a component thereof optionally selected from taurocholic acid (TCA), glycocholic acid (GCA) and cholic acid (CA).

In an embodiment, the bile acid is TBA.

In an embodiment, dilution correction of the LABW sample is standardized by the amount of total protein, albumin, or urea.

In an embodiment, the pepsinogen A4 cut-off level is about 0.3 ng/uL. In an embodiment, the pepsinogen A4 cut-off level is or about 0.312 ng/uL or about 0.313 ng/uL and a level of the pepsinogen A4 greater than the cut-off level is indicative the lung is likely to have significant aspiration, and a level of pepsinogen A4 equal to or less than the cut-off level is indicative the lung is unlikely to have significant aspiration, and wherein the cut-off level is calculated by instilling and suctioning 20 mL of normal saline.

In an embodiment, the bile acid cut-off level is about 1245 nM and a level of the TBA greater than the cut-off level is indicative the lung as likely to have significant aspiration and a level of TBA equal to or less than the cut-off level is indicative the lung is unlikely to have had significant aspiration, and wherein the cut-off level is calculated by instilling and suctioning 20 mL of normal saline.

In an embodiment, the method is carried out at point-of-care.

In an embodiment, the level or the presence or absence, of pepsinogen A4, and optionally the level of the bile acid, is detected by an immunoassay.

In an embodiment, the level or the presence or absence, of pepsinogen A4, and optionally the level of the bile acid, is detected by one or more of a Luminex® based assay, an enzyme-linked immunosorbent assay (ELISA), immunofluorescence, a radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, and any combination thereof.

The methods described herein can be used in general in assessing aspiration or diseases associated with aspiration, for example, aspiration pneumonia, cystic fibrosis, emphysema, and pulmonary fibrosis.

In a further aspect, the methods described herein can be used in general in assessing clinical airway infection in lung transplant recipients. In an embodiment, pepsinogen A4 is for use in post-transplant surveillance. In an embodiment, the surveillance comprises bronchoscopy and detecting the presence or absence of pepsinogen A4 in LABW. In an embodiment, detecting pepsinogen A4 in LABW is indicative of clinical airway infection. In an embodiment, detecting pepsinogen A4 in LABW is indicative of clinical airway infection and the recipient being at risk of or having gastric aspiration. In an embodiment, the clinical airway infection is treated by antibiotics, antivirals, or antifungals.

### III. Kits and Devices

Another aspect of the disclosure also includes kits and devices for the detection of the biomarkers of the disclosure, for example, pepsinogen A4, and optionally bile acid, that are used to measure the presence or absence of biomarker, or to measure biomarker levels.

In an embodiment, the devices and kits are suitable for use at point-of-care.

An aspect herein disclosed is a kit comprising one or more detection agents specific for pepsinogen A4 and optionally one or more detection agents specific for the level of bile acid, and instruction for use, the kit optionally further comprising one or more of a microtiter plate, an assay buffer, a wash buffer, a sample diluent, a standard diluent, a detection agent diluent, streptavidin-PE, a filter plate, a sealing tape, at least one standard, and any combination thereof, optionally for performing the methods disclosed herein. In an embodiment, the at least one standard is human pepsinogen A4. In an embodiment the at least one standard is bile acid. In an embodiment the at least one standard is human pepsinogen A4 and bile acid.

In an embodiment, the one or more detection agents are specific for pepsinogen A4. In an embodiment, the one or more detection agents are specific for bile acid. In an embodiment, the one or more detection agents are specific for pepsinogen A4 and bile acid.

The bile acid described herein can comprise total bile acid (TBA), or a component thereof selected from taurocholic acid (TCA), glycocholic acid (GCA), cholic acid (CA), and combinations thereof. In an embodiment, the bile acid is total bile acid (TBA). In an embodiment, the bile acid is total bile acid (TBA) or a component thereof selected from taurocholic acid (TCA), glycocholic acid (GCA), cholic acid (CA), and combinations thereof.

Pepsinogen A4 can be detected, for example, by antibodies that are specific for pepsinogen A4. In an embodiment, the detection agents specific for pepsinogen A4 are one or more antibodies.

In an embodiment, the detection agents specific for pepsinogen A4 is at least one monoclonal antibody. In an embodiment, the antibody comprises at least one of clone 021, clone 03, clone 06, clone 10, clone 961092, and clone 762015 from Novus Biologicals, and clone 762015 from R&D Systems.

In an embodiment, the detection agents specific for pepsinogen A4 is at least one polyclonal antibody. In another embodiment, the polyclonal antibody host is rabbit, goat, sheep, mouse, rat, guinea pig, hamster, chicken or llama.

In an embodiment, the detection agents specific for pepsinogen A4 are for the detection of protein.

In an embodiment, the one or more antibodies specific for pepsinogen A4 is conjugated, for example, conjugated to a fluorophore, biotin or HRP.

In an embodiment, the microtiter plate is polystyrene, polypropylene or polycarbonate.

In an embodiment, the microtiter plate is black, white or clear.

In an embodiment, the microtiter plate is conical (v-bottom), round (u-bottom), or flat bottom.

In an embodiment, the microtiter plate is a 96, 384 or 1536-well plate.

In an embodiment, the kit comprises an immunoassay for one or more biomarkers of the disclosure. In an embodiment, the kit comprises an immunoassay for pepsinogen A4. In an embodiment, the kit comprises an immunoassay for pepsinogen A4 and bile acid. Each kit comprises at least one detection antibody specific for one or more biomarker. For example, the antibody can be in the form of antibody coupled beads such as antibody coupled magnetic beads, or labelled antibodies, optionally comprised in a cartridge. In an embodiment, the kit further comprises one or more of a microtiter plate, a cartridge comprising one or more antibodies, standards, assay buffer, wash buffer, sample diluent, standard diluent, detection antibody diluent, streptavidin-PE, a filter plate and sealing tape, and any combination thereof. In an embodiment, the kit comprises detection antibodies or assays for detecting pepsinogen A4. In an embodiment, the kit comprises detection antibodies or assays for detecting pepsinogen A4 and bile acid. In an embodiment the kit comprises detection antibodies or assays for detecting two or more of pepsinogen A4 and bile acid e.g. two or more of bile acid (e.g. TCA, GCA, CA), and pepsinogen A4. In an embodiment, the kit comprises detection antibodies or assays for detecting TCA and GCA. In an embodiment, the kit comprises detection antibodies or assays for detecting bile acid TCA and CA. In an embodiment, the kit comprises detection antibodies or assays for detecting bile acid GCA and CA. In an embodiment, the kit comprises detection antibodies or assays for detecting bile acid (e.g. TCA, GCA, CA), and pepsinogen A4.

In an embodiment, the kits are for use for a method described herein.

Another aspect is a point-of-care (POC) device for detecting the level, or the presence or absence of pepsinogen A4, and optionally for detecting the level of bile acid, in a sample. The device comprises:
a housing,
a porous medium comprised in the housing, the porous medium comprising at a first end a sample portion and at a second end a testing portion,
a channel defined between the sample portion and the testing portion, the channel defining a flow path along which the sample passes and is drawn from the sample portion to the testing portion by capillary action, and
one or more reagents or detection agents for pepsinogen A4, and optionally one or more reagents or detection agents for bile acid, the one or more reagents or detection agents being capable of reacting with the pepsinogen A4 to generate a detection signal, and optionally one or more reagents or detection agents for bile acid.

In an embodiment, the one or more reagents or detection agent are in fluid communication with the testing portion or immobilized on the testing portion. In an embodiment, the detection signal is a visible color change or a fluorescent signal.

In an embodiment, a sample delivered into or on the sample portion migrates to the testing portion via capillary action.

In an embodiment, the one or more reagents or detection agents comprise pepsinogen A4. In an embodiment, the one or more reagents or detection agents comprise bile acid. In an embodiment, the one or more reagents or detection agents comprise pepsinogen A4 and bile acid.

The bile acid described herein can comprise total bile acid (TBA), or a component thereof selected from taurocholic acid (TCA), glycocholic acid (GCA), cholic acid (CA), and combinations thereof. In an embodiment, the bile acid is total bile acid (TBA). In an embodiment, the bile acid is total bile acid (TBA) or a component thereof selected from taurocholic acid (TCA), glycocholic acid (GCA), cholic acid (CA), and any combination thereof.

In an embodiment, the sample is a lung-derived fluid sample.

The lung-derived fluid sample can be sourced from a number of different types of samples, for example, it can be a bronchoalveolar lavage (BAL) sample, a bronchial wash sample, an exhaled breath condensate sample, or an ex vivo lung perfusion (EVLP) perfusate sample. In an embodiment, the lung-derived fluid sample is a BAL sample, a bronchial wash sample, an exhaled breath condensate sample, or an ex vivo lung perfusion (EVLP) perfusate sample. In an embodiment, the lung-derived fluid sample is a bronchoalveolar lavage (BAL) sample. In an embodiment, the lung-derived fluid sample is a bronchial wash sample. In an embodiment, the lung-derived fluid sample is an exhaled breath condensate sample. In an embodiment, the lung-derived fluid sample is an ex vivo lung perfusion (EVLP) perfusate sample. In an embodiment, the bronchial wash sample is a large airway bronchial wash (LABW) sample.

In an embodiment, the device is an immunoassay.

In an embodiment, the immunoassay is one or more of an enzyme-linked immunosorbent assay (ELISA), a multi-plex assay, an electrochemical assay, an enzymatic assay, a colorimetric assay, and any combination thereof.

In an embodiment, the devices described herein can also be described as lateral flow devices. Herein, the term “lateral flow device” generally refers to a device that includes one or more fluid channels, chambers or conduits that spontaneously drive a fluid across the device (e.g. by capillary force). Lateral flow device is well known in the art and it can be in a variety of formats such as sandwich format, competitive format, and multiplex format. Lateral flow device can also be used with a variety of affinity reagents, such as antibodies and aptamers (see e.g., Sajid M, Kawde A and Daud M (2015) Design, Formats and Applications of Lateral Flow Assays, J Saudi Chem Soc., 19, 689-705, and Bahad [symbol] r E B & Sezgintürk M K (2016) Lateral flow assays: Principles, designs and labels. Trends in Analytical Chemistry, 82, 286-306, the contents of which are incorporated by reference herein in their entireties). It is known in the art that sensitivity of a lateral flow device is affected by various factors such as pore size of the nitrocellulose membrane and flow rate, and experimental conditions can be optimized for lateral flow device performance (See Bahad [symbol] r, E. B., & Sezgintürk, M. K. (2016)). In an embodiment, the POC device comprises a lateral flow device wherein the lateral flow device has been optimized such that the level of pepsinogen A4 and optionally bile acid, is not detectable in samples from suitable transplant, but detectable in samples from poor transplant. In some embodiments, the level of pepsinogen A4 and optionally bile acid is not detectable in sample from suitable transplant, but detectable in samples from poor transplant. It can be understood that a lateral flow device for detecting or measuring pepsinogen A4 and optionally bile acid disclosed herein can be optimized based on those factors affecting sensitivity. The skilled person can also readily configure a lateral flow device that provides control signals for ease of use or to increase the accuracy of the device. In an embodiment, the lateral flow device comprises a control line for checking device function. In an embodiment, the lateral flow device comprises a control link for checking assay function.

The lateral flow device described herein is intended for rapid detection of the presence of an analyte in a test sample without the need for costly or sophisticated equipment. This device is useful in POC applications, as well as in laboratory testing or medical diagnostics. There are a number of configurations for a lateral flow device known to the person skilled in the art. For example, the lateral flow device described herein can comprise a sensor portion having an immobilized and stabilized sensor, and a testing portion having stabilized reporting solution. In an embodiment, the lateral flow device comprises a buffer portion for applying a running buffer, the buffer portion being connected through a flow channel to ii) a sensor portion for applying a sample, the sensor portion being connected through a flow channel to iii) a testing portion for indicating the presence, absence, or a range of levels of an analyte. In an embodiment, the running buffer comprises one or more reagents or detection agents. In an embodiment, the buffer portion is immobilized to a solid support. In an embodiment, the sensor portion is immobilized to a solid support. In an embodiment, the testing portion is immobilized to a solid support. In an embodiment, the testing portion comprises a plurality of testing zones. In an embodiment, the buffer portion, the sensor portion, and the testing portion are immobilized on a solid support. In an embodiment, the buffer portion comprises one or more reagents or detection agents for an analyte. In an embodiment, the sensor portion comprises one or more reagents or detection agents for an analyte. In an embodiment, the testing portion comprises one or more reagents or detection agents for an analyte. In an embodiment, the buffer portion comprises one or more analyte-specific antibodies. In an embodiment, the sensor portion comprises one or more analyte-specific antibodies. In an embodiment, the testing portion comprises one or more analyte-specific antibodies. In an embodiment, the analyte is pepsinogen A4. In an embodiment, the analyte is pepsinogen A4 and bile acid. In an embodiment, the one or more analyte-specific antibodies are coupled to a detectable label. In an embodiment, the one or more reagents or detection agents are capable of reacting with the analyte and/or the one or more analyte-specific antibodies to generate a detection signal. In an embodiment, the detection signal is a visible color change. In an embodiment, the detection signal is a fluorescent signal. In an embodiment, the color change or fluorescent signal is proportionate to the amount of pepsinogen A4. In an embodiment, the color change or fluorescent signal is proportionate to the amount of bile acid. In an embodiment, the color change or fluorescent signal is proportionate to the amount of pepsinogen A4 and bile acid. In an embodiment, the color change or fluorescent signal is titrated with the amount of pepsinogen A4. In an embodiment, the color change or fluorescent signal is titrated with the amount of bile acid. In an embodiment, the color change or fluorescent signal is titrated with the amount of pepsinogen A4 and bile acid. In an embodiment, the signal, optionally a fluorescent signal, instill is detected by a reader. The skilled person can recognize suitable fluorescent label for the generation of a fluorescent signal, and suitable reader for detecting the fluorescent signal.

In another embodiment, the lateral flow device comprises a sample portion and a testing portion. In an embodiment, the sample portion and the testing portion are connected through a flow channel. In an embodiment, the sample portion is for applying a running buffer and an analyte. In an embodiment, the sample portion is for applying a mixture comprising a running buffer and an analyte. In an embodiment, the lateral flow device comprises a sample portion for applying a mixture of a running buffer and an analyte, the sample portion being connected through a flow channel to a testing portion for indicating the presence, absence, or a range of levels of an analyte. In an embodiment, the sample portion is immobilized to a solid support. In an embodiment, the testing portion is immobilized to a solid support. In an embodiment, the sample portion and the testing portion are immobilized to a solid support. In an embodiment, the testing portion comprises a plurality of testing zones. In an embodiment, the sample portion comprises one or more reagents or detect agents for an analyte. In an embodiment, the testing portion comprises one or more reagents or detect agents for an analyte. In an embodiment, the sample portion comprises one or more analyte-specific antibodies. In an embodiment, the testing portion comprises one or more analyte-specific antibodies. In an embodiment, the sample portion and the testing portion each comprises one or more analyte-specific antibodies. In an embodiment, the analyte is pepsinogen A4. In an embodiment, the analyte is bile acid. In an embodiment, the analyte is pepsinogen A4 and bile acid. In an embodiment, the analyte is pepsinogen A4 and total bile acid. In an embodiment, the one or more analyte-specific antibodies are coupled to a detectable label. In an embodiment, the one or more reagents or detection agents are capable of reacting with the analyte and/or the one or more analyte-specific antibodies to generate a detection signal. In an embodiment, the detection signal is a visible color change. In an embodiment, the detection signal is a fluorescent signal. In an embodiment, the color change or fluorescent signal is proportionate to the amount of pepsinogen A4. In an embodiment, the color change or fluorescent signal is proportionate to the amount of bile acid. In an embodiment, the color change or fluorescent signal is proportionate to the amount of pepsinogen A4 and bile acid. In an embodiment, the color change or fluorescent signal is titrated with the amount of pepsinogen A4. In an embodiment, the color change or fluorescent signal is titrated with the amount of bile acid. In an embodiment, the color change or fluorescent signal is titrated with the amount of pepsinogen A4 and bile acid. In an embodiment, the fluorescent signal is detected by a reader. The skilled person can recognize suitable fluorescent label for the generation of a fluorescent signal, and suitable reader for detecting the fluorescent signal.

The lateral flow device described herein can involve the use of a conjugate comprising an antibody coupled to a detectable label, such as a colored particle such as a metal sol or colloid. In an embodiment, the detectable label is an enzymatic moiety. In an embodiment, the enzymatic moiety comprises urease, alkaline phosphatase, horseradish peroxidase, glucose oxidase, or β-galactosidase. In an embodiment, the enzymatic moiety is a pH changing enzyme. In an embodiment, the pH changing enzyme comprises urease, and the substrate comprises urea, and the color changing dye comprises bromothymol blue, phenol red, neutral red, cresol red, m-cresol purple, or o-cresolphthalein complexone. In an embodiment, the enzymatic moiety comprises horseradish peroxidase, and the substrate comprises 2,2′-Azinobis [3-ethylbenzothiazoline-6-sulfonic acid]-diammonium salt (ABTS), 3-amino-9-ethylcarbazole (AEC), 3,3-diaminobenzidine (DAB), 3,3′,5,5′-Tetramethylbenzidine (TMB), or Amplex Red. In an embodiment, urease catalyzes the conversion of urea to ammonia, which increases the pH of the solution, which can then be detected by a change in color of the color changing dye. In an embodiment, the metal sol or colloid is gold.

The conjugate can be in a form suitable for, e.g. sandwich or competitive format. When the lateral flow device relates to the sandwich format, it can involve a first antibody that is capable of binding to a first epitope on the analyte (e.g. pepsinogen A4, and optionally bile acid), and by which the first antibody is immobilized at the testing zone of the testing portion of the lateral flow device. Under this format, there is also a conjugate comprising a detectable label and a second antibody that is capable of binding to a second epitope on the analyte, and the binding of the conjugate to the analyte would form a complex. The formation of this complex is detected at the testing zone, where the complex reacts with the immobilized first antibody to form a “sandwich” of the first antibody, analyte, and conjugate (i.e. the second antibody coupled with a detectable label). This sandwich comprising the analyte is detected by visual observation of color development, as the sandwich is progressively produced at the testing zones where the conjugate-analyte complex is captured by the first antibody. As more conjugate is immobilized for the formation of the sandwich, the detectable labels aggregate at the testing zones and become visible because they are colored, or through an enzymatic reaction, indicating the presence of the analyte in the sample. The skilled person can also readily construct a device that produces a signal when the amount of the analyte is above a cut-off threshold described herein.

When the lateral flow device relates to the competitive format, it can involve a conjugate comprising an authentic sample of the analyte and a detectable label. The conjugate would be in competition with the analyte for binding to an analyte-specific antibody, which is immobilized at the testing zone. As the sample and the conjugate flow through the device and arrive at the testing zones, any analyte in the sample would compete with the conjugate for sites of attachment to the analyte-specific antibody. If no analyte is in the sample, the detectable labels aggregate at the testing zone, and the development of color, for instance because the detectable label is colored or through an enzymatic reaction, indicates the absence of detectable levels of the analyte in the sample. If the analyte is in the sample, the amount of conjugate which binds at the testing zone is reduced (i.e. competed out by the analyte), and no color, or a lighter color, is observed. The skilled person can also readily construct a device that produces a signal when the amount of the analyte is above a cut-off threshold described herein.

A number of materials are useful for making the lateral flow device described herein. The lateral flow device can have separated zones and flow channels. Such zones and flow channels can be created by wax on a nitrocellulose paper backed with a plastic sheet, i.e. wax acts as a uniform hydrophobic barrier for which the running buffer does not penetrate and the nitrocellulose paper acts to allow lateral flow of the running buffer. Methods for creating a hydrophobic barrier on a support layer are known to the person skilled in the art. The skilled person also recognizes that many alternatives to nitrocellulose paper are possible, for example, any material that allows flow could work, such as cellulose, or any other surface that supports capillary flow. Accordingly, in an embodiment, the lateral flow device comprises nitrocellulose paper, cellulose, or any surface that supports capillary flow. In an embodiment, the lateral flow device comprises nitrocellulose paper. In an embodiment, the lateral flow device comprises a polymer support layer. In an embodiment, the polymer support layer comprises a plastic sheet. In an embodiment, the lateral flow device comprises a hydrophobic material. In an embodiment, the hydrophobic material comprises wax. In an embodiment, the lateral flow device was printed by a hydrophobic material. In an embodiment, the lateral flow device was printed by wax.

In an embodiment, the lateral flow device described herein is for use in identifying the lung (e.g. the subject), optionally the transplanted lung or donor lung as likely to have gastric aspiration when the level of pepsinogen A4, and optionally bile acid, provides a different signal output as compared to the cut-off level, or unlikely to have gastric aspiration when the level of pepsinogen A4, and optionally bile acid provides a similar signal output as compared to a cut-off level, or identifying the transplanted lung as likely to have gastric aspiration when the level of pepsinogen A4, and optionally bile acid provides a similar signal output as compared to a cut-off level, or unlikely to have gastric aspiration when the level of pepsinogen A4, and optionally bile acid provides a different output as compared to a cut-off level.

In an embodiment, the lateral flow device described herein is for use in identifying the lung e.g. transplanted lung or donor lung as likely to have gastric aspiration when the level of pepsinogen A4, and bile acid, provides a different signal output as compared to the cut-off level, or unlikely to have gastric aspiration when the level of pepsinogen A4, and bile acid provides a similar signal output as compared to a cut-off level, or identifying the lung, e.g., transplanted lung as likely to have gastric aspiration when the level of pepsinogen A4, and bile acid provides a similar signal output as compared to a cut-off level, or unlikely to have gastric aspiration when the level of pepsinogen A4, and bile acid provides a different output as compared to a cut-off level.

In an embodiment, the device is for use in performing a method described herein.

The kits and devices described herein can be used in general in assessing aspiration or complications associated with aspiration, for example, aspiration pneumonia, or in patients having a lung disease such as cystic fibrosis, emphysema, and pulmonary fibrosis where aspiration can carry greater risk.

The above disclosure generally describes the present disclosure. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLE

### Example 1—Large Airway Pepsinogen A4 is a Specific Marker of Gastric Aspiration and Predicts Chronic Lung Allograft Dysfunction in Lung Transplant Recipients

### Materials and Methods

Single cell RNA-seq datasets of healthy lungs were queried for expression of pepsinogen C (PGC) and pepsinogen A4 (PGA4). Results were validated by qPCR in four human lung samples.

### LABW Sample Collection

As per the International Society for Heart and Lung Transplantation Consensus

Statement for the Standardization of Bronchoalveolar Lavage in Lung Transplantation, LABW was collected by instilling and suctioning 20 ml of normal saline through a flexible bronchoscope in the mainstem bronchus (Martinu, (2020)). Samples were then centrifuged for 20 min at 3184 g at 4° C., supernatants were separated and frozen at −80° C.

### Quantitative Real-Time PCR

RNA was isolated from LABW samples using a commercially available kit and was reverse transcribed to cDNA using techniques known in the art. Quantitative real-time PCR was performed on a thermocycler using a commercially available polymerase (e.g. TaqMan or SYBR Green). The qPCR assays described herein may be adapted to work using techniques known in the art, with commercially available kits and reagents and on most Real-Time PCR instruments.

### Protein Measurements

All samples were thawed at 4° C. overnight. Total pepsin and pepsinogen A4 were measured in large airway bronchial washings (LABW) of 61 LTx recipients and 5 patients pre- and post-anti-reflux surgery using enzyme linked immunosorbent assay (ELISA). PGC and PGA4 were measured using the ELISA (R&D Systems, DuoSet ELISA) as per manufacturer's instructions. Briefly, 96-well plates were prepared by applying the diluted capture antibody overnight at room temperature. Wells were then washed twice, blocked and washed three times to remove blocking buffer. Samples were diluted in the reagent diluent provided and applied to the wells for 2 hours at room temperature. Plates were washed three times to remove unbound sample. Detection antibody was added to each well and incubated for 2 hours at room temperature. Plates were washed three times to remove unbound detection antibody. Streptavidin-HRP was added to each well, covered and incubated at room temperature for 20 minutes. Plates were washed three times to remove residual streptavidin-HRP. Substrate solution was added to each well, covered and incubated at room temperature for 20 minutes. Stop solution was added to each well, and the optical density was determined using a microplate reader.

### Post-Transplant Outcome Definitions

Follow-up data on transplant recipients was obtained from electronic medical records and computerized databases.

Acute Rejection: Transbronchial biopsies were clinically graded for acute vascular (A-grade) and airway inflammation (B-grade) rejection as per the International Society of Heart and Lung Transplantation consensus guidelines (Stewart S, Fishbein M C, Snell G I, Berry G J, Boehler A, Burke M M, Glanville A, Gould F K, Magro C, Marboe C C, McNeil K D, Reed E F, Reinsmoen N L, Scott J P, Studer S M, Tazelaar H D, Wallwork J L, Westall G, Zamora M R, Zeevi A, Yousem S A. Revision of the 1996 working formulation for the standardization of nomenclature in the diagnosis of lung rejection. J Heart Lung Transplant December; 26 (12):1229-42 (2007), incorporated herein by reference). A and B scores were calculated by averaging A and B grades within the 7 months post-transplant period, respectively. Recipients with biopsies failing to meet minimum standard (AX and/or BX) and those with no biopsy within the 7 months post-transplant period were excluded from the analysis.

Infection Score is a measure of infection and is defined as the number of BAL samples positive for infectious pathogens divided by the total number of BAL samples available over a given time period.

PGD is defined by the presence of pulmonary edema on chest X-ray and the PaO2/FiO2 ratio according to the 2016 International Society for Heart and Lung Transplantation Primary Graft Dysfunction Definition (Snell G I, Yusen R D, Weill D, Strueber M, Garrity E, Reed A, Pelaez A, Whelan T P, Perch M, Bag R, Budev M, Corris P A, Crespo M M, Witt C, Cantu E, Christie J D. Report of the ISHLT Working Group on Primary Lung Graft Dysfunction, part I: Definition and grading-A 2016 Consensus Group statement of the International Society for Heart and Lung Transplantation. J Heart Lung Transplant October; 36(10):1097-1103 (2017), Incorporated herein by reference.)

Chronic lung allograft dysfunction (CLAD) is defined as a sustained and irreversible decline in forced expiratory volume in one second (FEV₁)≤80% (based on two FEV₁ values separated by at least three weeks) of the post-transplant baseline (defined as the average of the two highest post-transplant FEV₁ values at least three weeks apart in the absence of other confounding etiologies). Date of CLAD onset was defined as the date of the first FEV₁≤80% of baseline in accordance with consensus guidelines from the International Society for Heart and Lung Transplantation (Verleden G M, Glanville A R, Lease E D, Fisher A J, Calabrese F, Corris P A, Ensor C R, Gottlieb J, Hachem R R, Lama V, Martinu T, Neil D A H, Singer L G, Snell G, Vos R. Chronic lung allograft dysfunction: Definition, diagnostic criteria, and approaches to treatment-A consensus report from the Pulmonary Council of the ISHLT. J Heart Lung Transplant May; 38(5):493-503 (2019), incorporated herein by reference).).

### Statistical Analysis

Univariable Cox proportional hazard models were used to assess the association between LABW pepsinogen A4 and adverse outcomes, recipient time to CLAD, and retransplant/death.

### Results

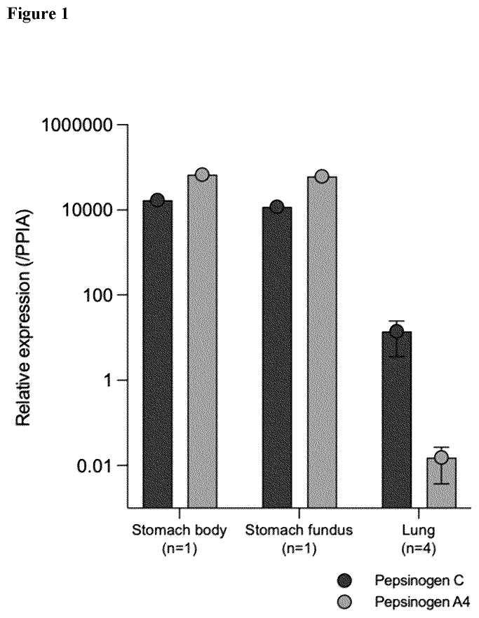

PGC is expressed in type II pneumocytes of healthy human lungs, while PGA4 is not expressed by any lung cell types. qPCR validated that PGC is expressed at significantly higher levels than PGA4 in human lungs (FIG. 1). Pepsin was detectable in 47/61 LABW samples, while pepsinogen A4 was only detectable in 7/61 samples. Anti-reflux surgery reduced LABW pepsinogen A4 levels (FIG. 2). LABW pepsin was not associated with time to CLAD or time to death/retransplant. However, LABW pepsinogen A4, but not total pepsin, was significantly associated with adverse outcomes (Table 1).

TABLE 1
Association between Pepsinogen A4 and recipient outcome

| | Time to CLAD | | Time to death/retransplant | |
|---|---|---|---|---|
| | Hazard Ratio | p- | HR | p- |
| | (HR) (95% CI) | value | (95%) CI | value |
| Total pepsin | 1.80 (0.61-5.26) | 0.29 | 1.01 (0.40-2.54) | 0.98 |
| Pepsinogen A4 | 1.81 (1.17-2.77) | 0.007 | 1.95 (1.27-2.98) | 0.002 |

These results show that pepsinogen A4 is a useful biomarker for assessing gastric aspiration in a transplanted lung, and for assessing the likelihood of developing an adverse outcome in a lung transplant recipient.

### Example 2A—Enzyme-Linked Immunosorbent Assay (ELISA) for Detecting Pepsinogen A4

Antibody-based detection of PGA4 to quantitively assess the level of PGA4 in recipient or donor LABW. The assay is based on detecting antigens of interest (e.g., PGA4) in LABW samples using antibodies that specifically bind to these antigens. The presence of antibodies can be quantitively assessed using the visible signals (absorbance) produced by enzymes linked to the antibodies. The quantification of PGA4, when used as a point-of-care device, is useful for obtaining rapid analytical results. This can facilitate the assessment of the suitability of donor lungs for transplantation.

As such, ELISA can be part of a point-of-care device such as a lateral flow device. One example of the lateral flow device is a simple and easy to use diagnostic kit with a sandwich ELISA lateral flow format for health care professional in a lung transplant team. In brief, the samples first come in touch with colored particles labeled with antibody raised against one or more bile acids (target analyte). The antibody can be used in the test line. The test line can also show a colorful band. The intensity of this band correlates with bile acid levels. This simple kit can come with a band color intensity meter to assist the judgment on PGA4 levels.

The second would be a quantitative device: In this device the intensity of the test line can be measured to determine the quantity of PGA4 in the sample. A lateral flow reader (for example, a handheld diagnostic device) can be used to provide a fully quantitative assay result. Using a suitable detection technology (complementary metal-oxide semiconductor (CMOS) or charge coupled device (CCD)) and unique wavelength of light for illumination, one can obtain a signal rich image from the actual test lines. With an image processing algorithm specifically designed for a PGA4 ELISA measurement, line intensities can then be converted to PGA4 concentrations. This quantitative lateral flow device can be used in the field hospitals and clinics, as well as diagnostic medical laboratories.

The same principles apply for a lateral flow device for detecting bile acid.

### Example 2B—Enzyme-Linked Immunosorbent Assay (ELISA) for Detecting Pepsinogen A4 (PGA4) in Lung Donor Bronchial Washings as a Marker of Aspiration and Predictor of Lung Transplant Recipient Outcomes

The utility of a commercially available PGA4 ELISA is tested to detect aspiration in transplanted lungs and lung donors and predict recipient outcomes in transplant lung recipients. Inventors measure PGA4 using a human PGA4 ELISA (R&D Biologics cat no: DY9105-05). This PGA4 ELISA kit applies the solid phase sandwich enzyme immunoassay technique using an anti-PGA4 capture and detection antibody and a streptavidin-HRP conjugate. The capture antibody is incubated on the uncoated plate overnight. Following the incubation period, the wells are decanted and washed three times, and then plates are blocked by adding the Reagent Diluent and incubated for two hours. Following the incubation period, the wells are decanted and washed three times. The bronchial wash sample is added to the wells and incubated for two hours. Following the incubation period, the wells are decanted and washed three times. The detection antibody is applied to the wells and incubated for two hours. Following the incubation period, the wells are decanted and washed three times. Streptavidin-HRP is then added to the wells and incubated, covered, for 20 minutes. Following the incubation period, the wells are decanted and washed three times. Substrate solution is added to each well and incubated for 20 minutes. The product of the enzyme-substrate reaction forms a blue colored complex. To stop the reaction a stop solution is added, which turns the solution yellow. The intensity of color is measured spectrophotometrically at 450 nm in a microplate reader. The intensity of the color is proportional to the PGA4 concentration. A standard curve is plotted relating the intensity of the color (O.D.) to the concentration of standards. The PGA4 concentration in each sample is interpolated from this standard curve.

### Example 3: LABW Pepsinogen A4 is Associated with Clinical Airway Infection

Post-transplant surveillance involving bronchoscopy was performed on lung transplant recipients. Detectable LABW pepsinogen A4 was found to be associated with increased frequency of clinical BAL infection in both the discovery and validation cohorts (Table 2 and Table 3). The discovery cohort was related to a retrospective case-control cohort to investigate the utility of measuring large airway bronchial washing (LABW) pepsin and pepsinogen A4 (Ref 23: Zhang et al., 2020), which included 61 adult bilateral LTRs at the Toronto Lung Transplant Program from 2010-2015 with post-transplant 24-hour pH/impedance testing and available post-transplant LABW/bronchoalveolar lavage (BAL) samples 60-120 days post-transplant. The validation cohort was related to a retrospective study cohort that included all first-time adult LTRs at the Toronto Lung Transplant Program from January 2010 to October 2017. Patients were excluded if they had <4 post-transplant pulmonary function tests or no LABW sample within 60-120 days post-transplant. During bronchoscopy, LABW samples were collected by instilling and suctioning 20 ml of saline in the mainstem bronchus. The bronchoscope was then wedged in a terminal bronchopulmonary segment and two fractions of BAL were obtained by instilling and suctioning two 50 mL aliquots of saline sequentially. LABW and BAL collection was defaulted to the right mainstem bronchus and right middle lobe, respectively, unless the LTR received a left single lung transplant or if there were localizing findings such as consolidation on imaging or localized secretions on airway examination. Samples were stored at 4° C. until they were centrifuged at 3184G at 4° C. Supernatants were separated and stored at −80° C. in the Toronto Lung Transplant Biobank (REB #11-0509). The results in Tables 2 and 3 demonstrate that pepsinogen A4 serves as a reliable biomarker for identifying lung transplant recipients who are at risk of gastric aspiration and also clinical airway infection.

TABLE 2
Patient demographics and bronchoscopy-based characteristics of lung transplant
recipients with and without detectable LABW pepsinogen A4 (PGA4)

| | PGA4 Detectable | PGA4 Undetectable | |
|---|---|---|---|
| | (n = 20) | (n = 180) | p-value |
| Transplant year | 2016 (2016-2016) | 2016 (2016-2016) | 0.52 |
| Recipient age | 60 (57-62) | 63 (57-67) | 0.29 |
| Recipient sex [Male, n (%)] | 9 (45) | 112 (62) | 0.13 |
| Transplant Type | | | |
| BLT, n (%) | 17 (85) | 148 (82) | 0.77 |
| Native Lung Disease, n (%) | | | |
| COPD | 4 (20) | 36 (20) | 0.70 |
| Cystic Fibrosis | 2 (10) | 27 (15) | |
| Pulmonary Fibrosis | 8 (40) | 82 (46) | |
| Other | 6 (30) | 35 (19) | |
| Canadian recipient urgency | | | |
| status, n (% )^{a} | | | |
| 1 | 3 (15) | 44 (24) | 0.14 |
| 2 | 7 (35) | 87 (48) | |
| 3 | 10 (50) | 49 (28) | |
| Donor Characteristics | | | |
| Age | 40 (43-53) | 39 (47-56) | 0.76 |
| Male, n (%) | 13 (65) | 108 (60) | 0.66 |
| DBD, n (%) | 14 (70) | 144 (80) | 0.29 |
| EVLP, n (%) | 7 (35) | 64 (36) | 0.96 |
| CMV Mismatch (donor | 4 (20) | 37 (21) | 0.99 |
| seropositive/recipient | | | |
| seronegative), n (%) | | | |

Bronchoscopy-based characteristics

| | | | |
|---|---|---|---|
| Days from transplantation | 92 (90-93) | 94 (91-96) | 0.92 |
| to bronchoscopy | | | |
| Bronchoscopy location, n (%) | | | |
| RML or RLL | 18 (90) | 152 (84) | 0.74 |
| Other | 2 (10) | 28 (16) | |
| Acute cellular rejection | | | |
| grade, n (%) | | | |
| A0: none | 13 (65) | 91 (50) | 0.42 |
| A1: minimal | 4 (20) | 31 (17) | |
| A2: mild | 2 (10) | 5 (3) | |
| AX: insufficient tissue or | 1 (5) | 53 (30) | |
| no biopsy performed | | | |
| Lymphocytic bronchiolitis | | | |
| grade, n (%) | | | |
| B0: none | 6 (30) | 62 (34) | 0.83 |
| B1R: minimal | 0 (0) | 1 (1) | |
| BX: insufficient tissue or | 14 (70) | 117 (65) | |
| no biopsy performed | | | |
| Acute rejection, n (%) ^{b} | 3 (15) | 25 (14) | 0.99 |
| Significant pathogens in | 8 (40) | 32 (18) | 0.03 |
| BAL, n (%) | | | |
| Clinical infection, n (%) ^{c} | 7 (25) | 24 (16) | 0.02 |
| BAL cytology findings, n (%) ^{d} | | | |
| Neutrophilic inflammation | 6 (30) | 33 (18) | 0.99 |
| Lymphocytic inflammation | 4 (20) | 19 (11) | |
| Eosinophilic inflammation | 0 (0) | 1 (0.5) | |

Abbreviations: PGA4—pepsinogen A4, BLT—bilateral lung transplant, COPD—chronic obstructive pulmonary disease, DBD—donation after brain death, EVLP—ex vivo lung perfusion, CMV—cytomegalovirus, BAL—bronchoalveolar lavage, RML—right middle lobe, RLL—right lower lobe
^{a}Canadian urgency status: 1 is assigned to patients with lowest acuity, while 3 designates patients with highest severity of disease on the waiting list.
^{b} Acute rejection - A1 or greater pathology score on biopsy and subsequently started on augmented immunosuppression
^{c}Clinical infection - positive BAL culture and subsequently started on antibiotics
^{d} Neutrophilic inflammation was defined as >3% neutrophils, lymphocytic inflammation as >15% lymphocytes, and eosinophilic inflammation as >1% eosinophils in the BAL

TABLE 3
Patient demographics and bronchoscopy-based characteristics of lung transplant
recipients in the discovery cohort with and without detectable LABW pepsinogen A4 (PGA4).

| | PGA4 Detectable | PGA4 Undetectable | |
|---|---|---|---|
| | (n = 7) | (n = 54) | p-value |
| Transplant year | 2013 (2013-2013) | 2013 (2013-2014) | 0.45 |
| Recipient age | 56 (56-64) | 59 (56-62) | 0.75 |
| Recipient sex [Male, n (%)] | 5 (71) | 30 (56) | 0.64 |
| Transplant Type | | | |
| BLT, n (%) | 6 (86) | 47 (87) | 0.92 |
| Native Lung Disease, n (%) | | | |
| COPD | 1 (14) | 12 (22) | 0.83 |
| Cystic Fibrosis | 1 (14) | 7 (13) | |
| Pulmonary Fibrosis | 3 (43) | 27 (50) | |
| Other | 2 (29) | 8 (15) | |
| Canadian recipient urgency | | | |
| status, n (%) ^{a} | | | |
| 1 | 1 (14) | 17 (31) | 0.27 |
| 2 | 5 (72) | 19 (36) | |
| 3 | 1 (14) | 18 (33) | |
| Donor Characteristics | | | |
| Age | 39 (48-67) | 35 (47-53) | 0.42 |
| Male, n (%) | 6 (86) | 34 (63) | 0.23 |
| DBD, n (%) | 6 (86) | 47 (87) | 0.92 |
| EVLP, n (%) | 1 (14) | 16 (30) | 0.66 |
| CMV Mismatch, n (%) | 1 (14) | 13 (24) | 0.99 |

Bronchoscopy-based characteristics

| | | | |
|---|---|---|---|
| Days from transplantation | 92 (91-93) | 93 (90-95) | 0.43 |
| to bronchoscopy | | | |
| Acute cellular rejection | | | |
| grade, n (%) | | | |
| A0: none | 3 (43) | 33 (61) | 0.25 |
| A1: minimal | 3 (43) | 9 (17) | |
| AX: insufficient tissue or | 1 (14) | 12 (22) | |
| no biopsy performed | | | |
| Lymphocytic bronchiolitis | | | |
| grade, n (%) | | | |
| B0: none | 2 (29) | 27 (50) | 0.09 |
| B1R: minimal | 1 (14) | 0 (0) | |
| BX: insufficient tissue or | 4 (57) | 27 (50) | |
| no biopsy performed | | | |
| Acute rejection, n (%) ^{b} | 1 (14) | 6 (11) | 0.99 |
| Significant pathogens in | 4 (57) | 7 (13) | 0.02 |
| BAL, n (%) | | | |
| Clinical infection, n (%) ^{c} | 3 (43) | 5 (9) | 0.04 |
| BAL cytology findings, n (%) ^{d} | | | |
| Neutrophilic inflammation | 3 (43) | 8 (15) | 0.10 |
| Lymphocytic inflammation | 0 (0) | 1 (2) | 0.99 |
| Eosinophilic inflammation | 1 (14) | 1 (2) | 0.22 |

Abbreviations: PGA4—pepsinogen A4, BLT—bilateral lung transplant, COPD—chronic obstructive pulmonary disease, DBD—donation after brain death, EVLP—ex vivo lung perfusion, CMV—cytomegalovirus, BAL—bronchoalveolar lavage
^{a} Canadian urgency status: 1 is assigned to patients with lowest acuity, while 3 designates patients with highest severity of disease on the waiting list
^{b} Acute rejection - A1 or greater pathology score on biopsy and subsequently started on augmented immunosuppression
^{c} Clinical infection - positive BAL culture and subsequently started on antibiotics
^{d} Neutrophilic inflammation was defined as >3% neutrophils, lymphocytic inflammation as >15% lymphocytes, and eosinophilic inflammation as >1% eosinophils in the BAL

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS FOR REFERENCES REFERRED TO IN THE SPECIFICATION

1. M. Cypel, J. C. Yeung, S. Keshavjee, Novel approaches to expanding the lung donor pool: donation after cardiac death and ex vivo conditioning. Clin Chest Med 32, 233-244 (2011).
2. M. K. Hsin, I. Iskender, D. Nakajima, M. Chen, H. Kim, P. R. dos Santos, J. Sakamoto, J. Lee, K. Hashimoto, C. Harmantas, D. Hwang, T. Waddell, M. Liu, S. Keshavjee, M. Cypel, Extension of donor lung preservation with hypothermic storage after normothermic ex vivo lung perfusion. J Heart Lung Transplant 35, 130-136 (2016).
3. T. N. Machuca, O. Mercier, S. Collaud, J. Tikkanen, T. Krueger, J. C. Yeung, M. Chen, S. Azad, L. Singer, K. Yasufuku, M. de Perrot, A. Pierre, T. K. Waddell, S. Keshavjee, M. Cypel, Lung transplantation with donation after circulatory determination of death donors and the impact of ex vivo lung perfusion. Am J Transplant 15, 993-1002 (2015).
4. D. Nakajima, M. Cypel, T. Martinu, M. Liu, S. Keshavjee, Donor bronchial wash bile acid and suitability of donor lungs for transplantation. J Heart Lung Transplant 37, 304-306 (2017).
5. D. Nakajima, M. Liu, A. Ohsumi, R. Kalaf, I. Iskender, M. Hsin, T. Kanou, M. Chen, B. Baer, R. Coutinho, L. Maahs, P. Behrens, S. Azad, T. Martinu, T. K. Waddell, J. F. Lewis, M. Post, R. A. W. Veldhuizen, M. Cypel, S. Keshavjee. Lung lavage and surfactant replacement during ex vivo lung perfusion for treatment of gastric acid aspiration-induced donor lung injury. J Heart Lung Transplant 36, 577-585 (2017).
6. M. Cypel, S. Keshavjee, The clinical potential of ex vivo lung perfusion. Expert Rev Respir Med 6, 27-35 (2012).
7. M. Cypel, M. Rubacha, J. Yeung, S. Hirayama, K. Torbicki, M. Madonik, S. Fischer, D. Hwang, A. Pierre, T. K. Waddell, M. de Perrot, M. Liu, S. Keshavjee, Normothermic ex vivo perfusion prevents lung injury compared to extended cold preservation for transplantation. Am J Transplant 9, 2262-2269 (2009).
8. M. Cypel, J. C. Yeung, S. Hirayama, M. Rubacha, S. Fischer, M. Anraku, M. Sato, S. Harwood, A. Pierre, T. K. Waddell, M. de Perrot, M. Liu, S. Keshavjee, Technique for prolonged normothermic ex vivo lung perfusion. J Heart Lung Transplant 27, 1319-1325 (2008).
9. M. Cypel, J. C. Yeung, M. Liu, M. Anraku, F. Chen, W. Karolak, M. Sato, J. Laratta, S. Azad, M. Madonik, C. W. Chow, C. Chaparro, M. Hutcheon, L. G. Singer, A. S. Slutsky, K. Yasufuku, M. de Perrot, A. F. Pierre, T. K. Waddell, S. Keshavjee, Normothermic ex vivo lung perfusion in clinical lung transplantation. N Engl J Med 364, 1431-1440 (2011).
10. J. C. Yeung, M. Cypel, E. Massad, S. Keshavjee, Ex vivo lung perfusion and reconditioning. Multimed Man Cardiothorac Surg 2011, Multimed Man Cardiothorac Surg 2011 Jan. 1; 2011 (418)
11. Kennedy, V. E., Todd, J. L. & Palmer, S. M. Bronchoalveolar Lavage as a Tool to Predict, Diagnose and Understand Bronchiolitis Obliterans Syndrome: Bronchoalveolar Lavage in BOS. American Journal of Transplantation 13, 552-561 (2013).
12. Young, L. R., Hadjiliadis, D., Davis, R. D. & Palmer, S. M. Lung Transplantation Exacerbates Gastroesophageal Reflux Disease. Chest 124, 1689-1693 (2003).
13. Hathorn, K. E., Chan, W. W. & Lo, W.-K. Role of gastroesophageal reflux disease in lung transplantation. WJT 7, 103 (2017).
14. King, B. J., Iyer, H., Leidi, A. A. & Carby, M. R. Gastroesophageal Reflux in Bronchiolitis Obliterans Syndrome: A New Perspective. The Journal of Heart and Lung Transplantation 28, 870-875 (2009).
15. Lawenko, R. M. A. & Lee, Y. Y. Evaluation of Gastroesophageal Reflux Disease Using the Bravo Capsule pH System. J Neurogastroenterol Motil 22, 25-30 (2015).
16. Parolo C et al., Tutorial: design and fabrication of nanoparticle-based lateral-flow immunoassays. Nat Protoc 15, 3788-3816 (2020).
17. Martinu T, Koutsokera A, Benden C, Cantu E, Chambers D, Cypel M, Edelman J, Emtiazjoo A, Fisher A J, Greenland J R, Hayes D Jr, Hwang D, Keller B C, Lease E D, Perch M, Sato M, Todd J L, Verleden S, von der Thusen J, Weigt S S, & Keshavjee S. International Society for Heart and Lung Transplantation consensus statement for the standardization of bronchoalveolar lavage in lung transplantation. J Heart Lung Transplant November; 39(11):1171-1190 (2020).
18. Sajid M, Kawde A and Daud M. Design, Formats and Applications of Lateral Flow Assays, J Saudi Chem Soc, 19, 689-705 (2015).
19. Bahad [symbol] r E B & Sezgintürk M K. Lateral flow assays: Principles, designs and labels. Trends in Analytical Chemistry, 82, 286-306 (2016).
20. Snell G I, Yusen R D, Weill D, Strueber M, Garrity E, Reed A, Pelaez A, Whelan T P, Perch M, Bag R, Budev M, Corris P A, Crespo M M, Witt C, Cantu E, Christie J D. Report of the ISHLT Working Group on Primary Lung Graft Dysfunction, part I: Definition and grading-A 2016 Consensus Group statement of the International Society for Heart and Lung Transplantation. J Heart Lung Transplant October; 36(10):1097-1103 (2017).
21. Verleden G M, Glanville A R, Lease E D, Fisher A J, Calabrese F, Corris P A, Ensor C R, Gottlieb J, Hachem R R, Lama V, Martinu T, Neil D A H, Singer L G, Snell G, Vos R. Chronic lung allograft dysfunction: Definition, diagnostic criteria, and approaches to treatment-A consensus report from the Pulmonary Council of the ISHLT. J Heart Lung Transplant May; 38(5):493-503 (2019).
22. Stewart S, Fishbein M C, Snell G I, Berry G J, Boehler A, Burke M M, Glanville A, Gould F K, Magro C, Marboe C C, McNeil K D, Reed E F, Reinsmoen N L, Scott J P, Studer S M, Tazelaar H D, Wallwork J L, Westall G, Zamora M R, Zeevi A, Yousem S A. Revision of the 1996 working formulation for the standardization of nomenclature in the diagnosis of lung rejection. J Heart Lung Transplant December; 26(12):1229-42 (2007).
23. Zhang C Y K, Ahmed M, Huszti E, Levy L, Hunter S E, Boonstra K M, et al. Bronchoalveolar bile acid and inflammatory markers to identify high-risk lung transplant recipients with reflux and microaspiration. J Heart Lung Transplant 2020; 39:934-944.

1. A method of assessing gastric aspiration in a lung, optionally a donor lung or a transplanted lung of a lung transplant recipient, the method comprising:
a) obtaining a lung-derived fluid sample from the lung such as a lung from a subject with cystic fibrosis, emphysema, or pulmonary fibrosis or the donor lung or the transplanted lung;
b) detecting a level, or the presence or absence, of pepsinogen A4 in the lung-derived fluid sample, and optionally detecting a level of a bile acid in the lung-derived fluid sample;
c) identifying the lung as i) likely to have gastric aspiration when pepsinogen A4 is present in the lung-derived fluid sample, or the level of pepsinogen A4 is greater than the pepsinogen A4 cut-off level in the lung-derived fluid sample, and optionally when the level of the bile acid is greater than the bile acid cut-off level in the lung-derived fluid sample, or ii) unlikely to have gastric aspiration when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample, or optionally iii) identifying the subject as having or likely to develop aspiration pneumonia, or having or likely to develop complications associated with gastric aspiration in the subject with cystic fibrosis, emphysema, or pulmonary fibrosis;
optionally wherein the bile acid cut-off level is determined by calculating in a population of donor lungs an average bile acid cut-off level above which at least 90% of the donor lungs were identified as having gastric aspiration;
d) optionally,
i) when pepsinogen A4 is present in the lung-derived fluid sample from the transplanted lung, or the level of pepsinogen A4 is greater than the pepsinogen A4 cut-off level in the lung-derived fluid sample from the transplanted lung, and optionally when the level of the bile acid in the lung-derived fluid sample from the transplanted lung is greater than the bile acid cut-off level, the method described herein further comprises treating the lung to reduce the likelihood of aspiration damage;
ii) when pepsinogen A4 is present in the lung-derived fluid sample from the donor lung, or the level of pepsinogen A4 is greater than the pepsinogen A4 cut-off level in the lung-derived fluid sample from the donor lung, and optionally when the level of the bile acid in the lung-derived fluid sample from the donor lung is greater than the bile acid cut-off level, the method described herein further comprises treating the donor lung to reduce the likelihood of aspiration damage;
iii) when pepsinogen A4 is present in the lung-derived fluid sample from the donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises performing EVLP on the donor lung to confirm lung function prior to transplantation;
iv) when pepsinogen A4 is present in the lung-derived fluid sample from the donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises treating the donor lung with surfactant;
v) when pepsinogen A4 is present in the lung-derived fluid sample from the donor lung, or the level of pepsinogen A4 from the lung-derived fluid sample from the donor lung is greater than the pepsinogen A4 cut-off level, and optionally when the level of the bile acid in the lung-derived fluid sample from the donor lung is greater than the bile acid cut-off level, the method described herein further comprises declining the donor lung for transplantation; or
vi) when pepsinogen A4 is present in the lung-derived fluid sample from the lung, or the level of pepsinogen A4 from the lung-derived fluid sample from the subject lung is greater than the pepsinogen A4 cut-off level, and optionally when the level of the bile acid in the lung-derived fluid sample from the subject lung is greater than the bile acid cut-off level, the method described further comprises treating the subject with complications associated with gastric aspiration.

2. The method of claim 1, wherein the lung-derived fluid sample is a bronchoalveolar lavage (BAL) sample, a bronchial wash sample, an exhaled breath condensate sample, or an ex vivo lung perfusion (EVLP) perfusate sample.

3. The method of claim 2, wherein the bronchial wash sample is large airway bronchial wash (LABW) sample.

4. The method of any one of claims 1 to 3, comprising detecting the level of a bile acid in the lung-derived fluid sample, wherein the bile acid is total bile acid (TBA) or a component thereof optionally selected from taurocholic acid (TCA), glycocholic acid (GCA) and cholic acid (CA).

5. The method of claim 4, wherein the bile acid is TBA.

6. The method of claim 3, wherein dilution correction of the LABW sample is standardized by the amount of total protein, albumin, or urea.

7. The method of claim 3, wherein the pepsinogen A4 cut-off level is about 0.3 ng/uL and a level of the pepsinogen A4 greater than the cut-off level is indicative the donor lung or transplanted lung is likely to have significant aspiration, and a level of pepsinogen A4 equal to or less than the cut-off level is indicative the donor lung or transplanted lung is unlikely to have significant aspiration, and wherein the cut-off level is calculated by instilling and suctioning 20 mL of normal saline.

8. The method of claim 5, wherein the bile acid cut-off level is about 1245 nM and a level of the TBA greater than the cut-off level is indicative the donor lung as likely to have significant aspiration and a level of TBA equal to or less than the cut-off level is indicative the donor lung is unlikely to have had significant aspiration, and wherein the cut-off level is calculated by instilling and suctioning 20 mL of normal saline.

9. The method of any one of claims 1 to 8, wherein the method is carried out at point-of-care.

10. The method of any one of claims 1 to 9, wherein the level or the presence or absence, of pepsinogen A4, and optionally the level of the bile acid, is detected by an immunoassay.

11. The method of any one of claims 1 to 9, wherein the level or the presence or absence, of pepsinogen A4, and optionally the level of the bile acid, is detected by one or more of a Luminex® based assay, an enzyme-linked immunosorbent assay (ELISA), immunofluorescence, a radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, and any combination thereof.

12. A method of assessing the likelihood of developing an adverse outcome in a lung transplant recipient, the method comprising:
a) obtaining a lung-derived fluid sample from a donor lung or a transplanted lung from the lung transplant recipient;
b) detecting a level, or the presence or absence, of pepsinogen A4 in the lung-derived fluid sample, and optionally detecting a level of a bile acid in the lung-derived fluid sample;
c) identifying the lung transplant recipient as i) likely to develop the adverse outcome when pepsinogen A4 is present in the lung-derived fluid sample, or the level of pepsinogen A4 is greater than the pepsinogen A4 cut-off level in the lung-derived fluid sample, and optionally when the level of the bile acid is greater than the bile acid cut-off level in the lung-derived fluid, or ii) unlikely to develop the adverse outcome when pepsinogen A4 is absent in the lung-derived fluid sample, or the level of pepsinogen A4 is equal or less than the pepsinogen A4 cut-off level in the lung-derived fluid sample, and optionally when the level of the bile acid is equal or less than the bile acid cut-off level in the lung-derived fluid sample,
optionally wherein the bile acid cut-off level is determined by calculating in a population of donor lungs an average bile acid cut-off level above which at least 90% of the donor lungs were identified as having gastric aspiration; and
d) optionally,
i) when pepsinogen A4 is present in the lung-derived fluid sample from the transplanted lung, or the level of pepsinogen A4 is greater than the pepsinogen A4 cut-off level in the lung-derived fluid sample from the transplanted lung, and optionally when the level of the bile acid in the lung-derived fluid sample from the transplanted lung is greater than the bile acid cut-off level, the method further comprises treating the lung transplant recipient to prevent the adverse outcome;
ii) when pepsinogen A4 is present in the lung-derived fluid sample from the donor lung, or the level of pepsinogen A4 is greater than the pepsinogen A4 cut-off level in the lung-derived fluid sample from the donor lung, and optionally when the level of the bile acid in the lung-derived fluid sample from the donor lung is greater than the bile acid cut-off level, the method further comprises treating the lung transplant recipient to prevent the adverse outcome;
iii) when pepsinogen A4 is present in the lung-derived fluid sample from the donor lung, or the level of pepsinogen A4 is greater than the pepsinogen A4 cut-off level in the lung-derived fluid sample from the donor lung, and optionally when the level of the bile acid in the lung-derived fluid sample from the donor lung is greater than the bile acid cut-off level, the method further comprises treating the donor lung with surfactant to prevent the adverse outcome; or
iv) when pepsinogen A4 is present in the lung-derived fluid sample from the donor lung, or the level of pepsinogen A4 is greater than the pepsinogen A4 cut-off level in the lung-derived fluid sample from the donor lung, and optionally when the level of the bile acid in the lung-derived fluid sample from the donor lung is greater than the bile acid cut-off level, the method further comprises declining the donor lung for transplantation.

13. The method of claim 12, wherein the adverse outcome is gastroesophageal reflux disease (GERD), chronic lung allograft dysfunction (CLAD), or death.

14. The method of claim 13, wherein the recipient identified as likely to develop the adverse outcome of GERD is selected for anti-reflux surgery or medical reflux medications.

15. The method of claim 13, wherein the anti-reflux surgery is Nissen fundoplication or Toupet fundoplication.

16. The method of claim 13, wherein the recipient identified as likely to develop the adverse outcome of CLAD or death is selected for retransplant.

17. The method of claim 16, wherein the retransplanted recipient is assessed for the likelihood of developing an adverse outcome.

18. The method of any one of claims 12 to 17, wherein the lung-derived fluid sample is a bronchoalveolar lavage (BAL) sample, a bronchial wash sample, an exhaled breath condensate sample, or an ex vivo lung perfusion (EVLP) perfusate sample.

19. The method of any one of claims 12 to 17, wherein the lung-derived fluid sample is a large airway bronchial wash (LABW) sample.

20. The method of any one of claims 12 to 19, comprising detecting the level of a bile acid in the lung-derived fluid sample, wherein the bile acid is total bile acid (TBA) or a component thereof optionally selected from taurocholic acid (TCA), glycocholic acid (GCA) and cholic acid (CA).

21. The method of claim 20, wherein the bile acid is TBA.

22. The method of claim 19, wherein dilution correction of the LABW sample is standardized by the amount of total protein, albumin, or urea.

23. The method of claim 19, wherein the pepsinogen A4 cut-off level is about 0.3 ng/uL and a level of the pepsinogen A4 greater than the cut-off level is indicative the donor lung or transplanted lung is likely to have significant aspiration, and a level of pepsinogen A4 equal to or less than the cut-off level is indicative the donor lung or transplanted lung is unlikely to have significant aspiration, and wherein the cut-off level is calculated by instilling and suctioning 20 mL of normal saline.

24. The method of claim 21, wherein the bile acid cut-off level is about 1245 nM and a level of the TBA greater than the cut-off level is indicative the donor lung as likely to have significant aspiration and a level of TBA equal to or less than the cut-off level is indicative the donor lung is unlikely to have had significant aspiration, and wherein the cut-off level is calculated by instilling and suctioning 20 mL of normal saline.

25. The method of any one of claims 12 to 24, wherein the method of assessing is carried out at point-of-care.

26. The method of any one of claims 12 to 25, wherein the level, or the presence or absence, of pepsinogen A4, and optionally the level of the bile acid, is detected by an immunoassay.

27. The method of any one of claims 12 to 25, wherein the level, or the presence or absence, of pepsinogen A4, and optionally the level of the bile acid, is detected by one or more of a Luminex® based assay, an enzyme-linked immunosorbent assay (ELISA), immunofluorescence, a radioimmunoassay, dot blotting, FACS, a protein microarray, immunoprecipitation followed by SDS-PAGE, immunocytochemistry, a simple Plex assay, a multiplex assay, mass spectrometry, an electrochemical assay, an enzymatic assay, a colorimetric assay, and any combination thereof.

28. A kit comprising one or more detection agents specific for pepsinogen A4, and optionally one or more detection agents specific for the level of the bile acid, and instruction for use, the kit optionally further comprising one or more of a microtiter plate, an assay buffer, a wash buffer, a sample diluent, a standard diluent, a detection agent diluent, streptavidin-PE, a filter plate, a sealing tape, at least one standard, and any combination thereof, optionally for performing the method of any one of claims 1 to 10, and 12 to 26.

29. A point-of-care (POC) device for detecting the level, or presence or absence, of pepsinogen A4, and optionally for detecting the level of a bile acid, in a sample, comprising:
a housing;
a porous medium comprised in the housing, the porous medium comprising at a first end a sample portion and at a second end a testing portion;
a channel defined between the sample portion and the testing portion, the channel defining a flow path along which the sample passes and is drawn from the sample portion to the testing portion by capillary action; and
one or more reagents or detection agents for pepsinogen A4, and optionally one or more reagents or detection agents for bile acid, optionally in fluid communication with the testing portion or immobilized on the testing portion, the one or more reagents or detection agents being capable of reacting with the pepsinogen A4 to generate a detection signal, and optionally one or more reagents or detection agents for bile acid, optionally a visible color change or a fluorescent signal.

30. The device of claim 29, comprising a plurality of flow paths through which the sample passes and which distributes the sample to a plurality of testing zones within the testing portion.

31. The device of claim 29, wherein each of the testing zones comprises a different reagent or detection agent.

32. The device of any one of claims 29 to 31, wherein the level, or the presence or absence, of pepsinogen A4, and optionally the level of the bile acid, is detected by an immunoassay.

33. The device of any one of claims 29 to 31, wherein the level, or the presence or absence, of pepsinogen A4, and optionally the level of the bile acid, is detected by an immunoassay, comprising one or more of an enzyme-linked immunosorbent assay (ELISA), a multiplex assay, an electrochemical assay, an enzymatic assay, a colorimetric assay, and any combination thereof.

34. The device of any one of claims 29 to 33, wherein the device is a lateral flow device.

35. The device of any one of claims 29 to 34, wherein the fluorescent signal is detected by a reader.

36. The device of any one of claims 29 to 34 for carrying out a method for any one of claims 1 to 27.

37. Use of the device of any one of claims 29 to 36 for performing the method of assessing of any one of claims 1 to 27.

38. A method of assessing gastric aspiration of a subject comprising:
a) obtaining a lung-derived fluid sample;
b) detecting a level, or the presence or absence, of pepsinogen A4 in the lung-derived fluid sample, and optionally detecting a level of a bile acid in the lung-derived fluid sample;
c) identifying a lung as i) likely to have gastric aspiration when pepsinogen A4 is present in the lung-derived fluid sample, or the level of pepsinogen A4 is greater than a pepsinogen A4 cut-off level in the lung-derived fluid sample, and optionally when the level of the bile acid is greater than a bile acid cut-off level in the lung-derived fluid sample, or ii) unlikely to have gastric aspiration when pepsinogen A4 is absent in the lung-derived fluid sample, or the level of pepsinogen A4 is equal or less than a pepsinogen A4 cut-off level in the lung-derived fluid sample, and optionally when the level of the bile acid is equal or less than a bile acid cut-off level in the lung-derived fluid sample, or optionally iii) identifying the subject as having or likely to develop aspiration pneumonia, or having or likely to develop complications associated with gastric aspiration in a subject with cystic fibrosis, emphysema, or pulmonary fibrosis;
optionally wherein the bile acid cut-off level is determined by calculating in a population of healthy lungs an average bile acid cut-off level above which at least 90% of the lungs were identified as having gastric aspiration.
